(19) United States
(12) Patent Application Publication
(10) Pub. No.: US 2026/0053664 A1
(43) Pub. Date: Feb. 26, 2026

(54) DEVICES AND METHODS FOR INJECTING SUBSTANCES AND/OR INTRODUCING IMPLANTS TOWARDS A TARGET SITE IN A BODY TISSUE

(71) Applicant: SUPRA MEDICAL LTD, Yokneam (IL)

(72) Inventors: Nir SINAI, Alon Hagalil (IL); Carmel HADAR, Aloney Habashan (IL); Doron SMULIAN, Haifa (IL)

(21) Appl. No.: 19/378,921

(22) Filed: Nov. 4, 2025

Related U.S. Application Data
(63) Continuation-in-part of application No. 18/772,181, filed on Jul. 14, 2024.
Application No. 18/772,181 is a continuation-in-part of application No. PCT/IB2023/050364, filed on Jan. 16, 2023.
(60) Provisional application No. 63/300,272, filed on Jan. 18, 2022.
(60) Provisional application No. 63/527,099, filed on Jul. 17, 2023.

(51) Int. Cl. A61F 9/00

(52) U.S. Cl. CPC A61F 9/0017

(57) ABSTRACT

An injection device has a longitudinal axis X that defines distal and proximal axial directions and includes a needle formed from relative rigid and inflexible material. The device also includes an abutment member at its distal side and the needle is movable to project in a distal direction beyond the abutment member along an axis T that is inclined relative to the longitudinal axis X.

### TECHNICAL FIELD

Embodiments of the invention relate to devices and methods for injecting substances (e.g. medication substances) and/or introducing implants towards a target site in a body tissue, for example beneath a tissue layer and/or towards a space between two tissue layers.

### BACKGROUND

Medication substances, such as drugs protein based substances (and the like) may be injected by physicians towards various target sites within the body. Some examples may include target sites between the dermis and the hypodermis, muscles below the facial skin (or the like). A medication substance such as botulinum toxin injected for example towards muscles under facial wrinkles is used for relaxing those muscles and smoothing the overlying skin. In some cases, it may be desirable to introduce other types of substances into specific target sites within the body, for example, to fill a space at the site, potentially in preparation for subsequent procedures such as implant placement.

Target sites within the eye provide a further example. Ophthalmic drug delivery for example is typically administrated by injecting such medication substances into the eye with a needle, and has been found to be a useful method for treating many retinal diseases, such as diabetic retinopathy, neovascular age-related macular degeneration (AMD), retinal vein occlusion (and the like).

This method is advantageous in providing a confined delivery of medications to a targeted site, as the needle can directly pass through the anatomical eye barrier (e.g. cornea, conjunctiva), while in turn minimizing unintended drug effects to other body tissues.

The suprachoroidal space (SCS) is one example of a site within the eye that can be targeted in an ophthalmic drug delivery procedure. The SCS is a space that can be formed between the sclera and the choroid, and therefore can be used for targeting e.g. the choroid, retinal pigment epithelium and retina, while substantially avoiding penetration of such medication substances to other regions of the eye.

In some situations, introducing implants to various target sites within the body can also be beneficial. These implants may include relatively thin, string-like structures capable of passing through a needle for minimally invasive implantation. In certain cases, such implants may serve purposes such as slow drug delivery (e.g. for controlled, localized drug release over time) or other medical applications, and typically may formed from biodegradable materials such as polymers or the like.

### SUMMARY

The following embodiments and aspects thereof are described and illustrated in conjunction with systems, tools and methods which are meant to be exemplary and illustrative, not limiting in scope.

In a broad aspect, various injection device embodiments of the present invention may be defined as being suitable for injecting medication substances towards a target site within, in-between and/or beneath a tissue layer.

The examples mainly discussed herein may be seen relating to ophthalmic injection devices, which are suitable for injecting medication substances such as drugs towards a target site within the eye, however the majority of the discussed embodiments may be also suitable for administrating medication substances in relation to other body tissues, such as towards skin layers, muscles below the facial skin (or the like).

In an embodiment there is provided an injection device having a longitudinal axis X generally defining distal and proximal axial directions and comprising a needle formed from relative rigid and inflexible material, the injection device further comprising an abutment member at its distal side and the needle being movable to project in a distal direction beyond the abutment member along an axis T that is inclined relative to the longitudinal axis X.

In a further embodiment there is provided a method for administrating a medication substance into a target layer within a body tissue that comprises the steps of: providing an injection device having a longitudinal axis X generally defining distal and proximal axial directions, the device comprising a needle formed from relative rigid and inflexible material and an abutment member at its distal end comprising an abutment face, engaging an outer side of the body tissue with the abutment face, and then urging the needle to penetrate into the body tissue towards the targeted layer along an axis T that is inclined to axis X.

And in yet a further embodiment there is provided an injection device comprising a needle, an abutment face and an opening at the abutment face, wherein the needle being formed from relative rigid and inflexible material and being movable to protrude beyond the abutment face through the opening along an axis T that is inclined relative to a tangent to the abutment face at the opening or to a plane passing through the abutment face at the opening.

Possibly, the abutment face is at least partially concave and the opening is comprised at the concave portion of the abutment face.

In yet a further embodiment there is provided an injection device having a longitudinal axis X generally defining distal and proximal axial directions, the injection device comprising: a needle formed from relative rigid and inflexible material, an actuating mechanism, and an abutment member at its distal side, wherein the needle being movable to project in a distal direction beyond the abutment member along an axis T that is inclined relative to the longitudinal axis X, and wherein the actuating mechanism comprising an actuator that when moved along axis X is configured to urge movement of the needle along axis T.

And in yet a further embodiment there is provided an injection device comprising two separate main members connected by a connecting tubing, a first one of the main members being a syringe hub member and a second one of the main members being an injector member, the syringe hub member being configured to connect to a syringe, and the injector member comprising a needle, an abutment face and an opening at the abutment face, wherein the needle being formed from relative rigid and inflexible material and being configured to protrude beyond the abutment face through the opening along an axis T that is inclined relative to a tangent to the abutment face at the opening or to a plane passing through the abutment face at the opening.

In the present disclosure, it is to be understood that use of the injection device embodiments disclosed herein, typically includes a preparation step of ensuring that substantially no air bubbles are present in liquid medication or substances intended to be administered by the injection device and its syringe.

This may be accomplished by slowly pushing the syringe's plunger to expel air bubbles and a small amount of liquid out of the tip of the injection device's needle, to ensure that the syringe and liquid passages leading to the needle's tip are filled with substantially only the medication to be administered.

In addition to the exemplary aspects and embodiments described above, further aspects and embodiments will become apparent by reference to the figures and by study of the following detailed descriptions.

### BRIEF DESCRIPTION OF THE FIGURES

Exemplary embodiments are illustrated in referenced figures. It is intended that the embodiments and figures disclosed herein are to be considered illustrative, rather than restrictive. The invention, however, both as to organization and method of operation, together with objects, features, and advantages thereof, may best be understood by reference to the following detailed description when read with the accompanying figures, in which:

FIG. 1A schematically shows an injection device in accordance with various embodiments of the present invention being used for administrating a substance towards a target site within a patient's body tissue, in this example within a skin tissue;

FIG. 1B schematically shows an injection device in accordance with various embodiments of the present invention being used for administrating a substance towards a target site within a patient's eye, hence being embodied in the example as an ophthalmic injection device;

FIGS. 2A and 2B schematically show respective non-exploded and partial exploded perspective side views of an embodiment of an injection device generally similar to the ones seen in FIGS. 1A and 1B;

FIGS. 3A and 3B schematically show a perspective side view and a top view of the injection device seen in FIGS. 2A and 2B, with the top view of FIG. 4 showing the injection device targeting an eye of the patient;

FIGS. 4A and 4B schematically show stopper and a lower side of a syringe of an embodiment of the injection device;

FIGS. 5A and 5B schematically show respective bottom views of an embodiment of an injection device with the view of FIG. 6B being shown in partial cross section;

FIG. 6A to 6C schematically show a terminal region of a needle member of an injection device in accordance with various embodiments of the present invention, during different stages of penetration into a tissue layer;

FIGS. 7A to 7C schematically show assembled and partial exploded views of an injection device embodiment generally similar to the one shown in the former figures;

FIG. 8 schematically shows a distal region of an injection device embodiment;

FIGS. 9A to 9G schematically show a possible embodiment of an injection device with a needle formed from relative rigid and inflexible material;

FIGS. 10A to 10C schematically show a possible embodiment of an injection device being divided into two separate main members;

FIGS. 11A and 11B schematically show an embodiment of an injection device, such as the one seen e.g. in FIGS. 10, 12 to 14 and 16—being positioned to inject substances towards posterior and latitudinal directions of an eye, respectively;

FIGS. 12A to 12C schematically show another possible embodiment of an injection device divided into two separate main members;

FIG. 13 schematically shows yet another possible embodiment of an injection device divided into two separate main members during different stages of operation;

FIG. 14 schematically shows a different view of the injection device of FIG. 13;

FIG. 15 schematically shows a perspective view of a safety catch possibly used in an injection device such as that seen in FIGS. 13 and 14;

FIG. 16 schematically shows an injection device in accordance with various embodiments of the present invention being held by a user of the device;

FIG. 17 schematically shows an embodiment of an injection device suitable for administrating a substance and/or introducing an implant towards a target site within a patient's body tissue;

FIGS. 18A to 18C schematically show an injection device such as the one seen in FIG. 17, during different stages of a minimally invasive procedure;

FIGS. 19A and 19B schematically show another embodiment of an injection device suitable for administrating a substance and/or introducing an implant towards a target site within a patient's body tissue;

FIGS. 20A and 20B schematically show yet another embodiment of an injection device suitable for administrating a substance and/or introducing an implant towards a target site within a patient's body tissue;

FIGS. 21A and 21B schematically show an embodiment of an injection device suitable for administrating a substance and/or introducing an implant towards a target site within a patient's body tissue, which includes a generally laterally extending implant introducer;

FIGS. 22A and 22B schematically show an embodiment of an injection device suitable for administrating a substance and/or introducing an implant towards a target site within a patient's body tissue, which includes a generally axially extending implant introducer;

FIG. 23A schematically shows an embodiment of an injection device generally similar to that in FIG. 22, which includes an implant loading member;

FIG. 23B schematically shows the implant loading chamber seen during different stages of inserting an implant therein; and

FIG. 24 schematically shows yet another embodiment of an injection device suitable for administrating a substance and/or introducing an implant towards a target site within a patient's body tissue.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated within the figures to indicate like elements.

### DETAILED DESCRIPTION

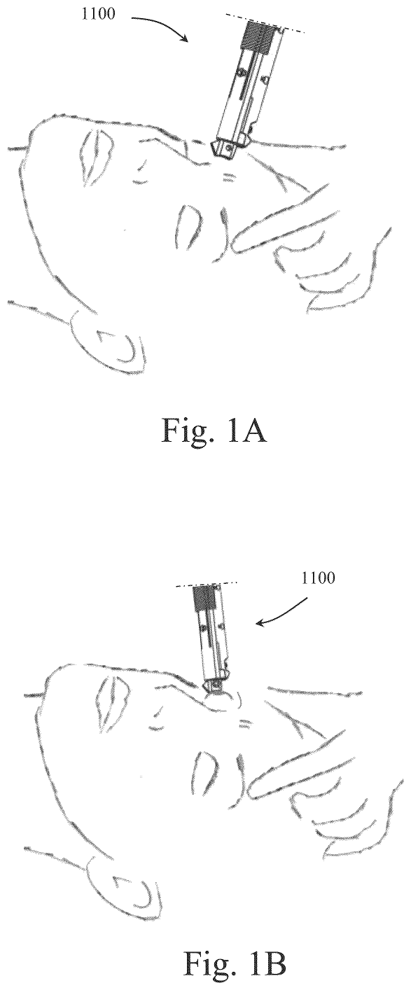

Attention is drawn to FIG. 1A schematically showing an injection device 1100 in accordance with various embodiments of the present invention being used for administrating a substance towards a target site within a patient's body tissue, in this example within a skin tissue. In one example the administered substance is a medication substance.

Examples of such target sites within a skin tissue may include sites between the dermis and the hypodermis, muscles below the facial skin (or the like). A medication substance such as botulinum toxin injected e.g. towards muscles under facial wrinkles can be used for relaxing those muscles and smoothing the overlying skin.

Attention is additionally drawn to FIG. 1B schematically showing an injection device (similar to device 1100 seen in FIG. 1A and hence being indicated with same numeral). Injection device 1100 in accordance with various embodiments of the present invention may be used for administrating a substance towards a target site within a patient's eye, hence being embodied in this example as an ophthalmic injection device.

It is noted that in a broad aspect, various injection device embodiments of the present invention that are disclosed herein—may be defined as being suitable for injecting substances towards a variety of target sites within, in-between and/or beneath one or more body tissue layers. In another broad aspect, certain injection device embodiments described herein may be designed substantially exclusively for introducing implants towards a target site, while others may be configured for both implant delivery and substance injection, potentially to similar target sites.

It is noted that while the examples mainly discussed herein below may be seen as relating to ophthalmic injection devices, which are suitable for injecting substances such as drugs towards a target site within the eye, the majority of the discussed embodiments may be also suitable for administrating substances in relation to other body tissues, such as towards skin layers, muscles below the facial skin (or the like).

Due to the costly nature of e.g. medication substances that may be used in such procedures and, e.g. in the case of an ophthalmic injection device, due to the widths and dimensions of the eye layers that may be targeted (which may be in the range of about 100 micron in width in certain cases), design considerations of the various injection device embodiments disclosed herein may be tailored for accurate and precise targeting of such target sites with the required substance doses.

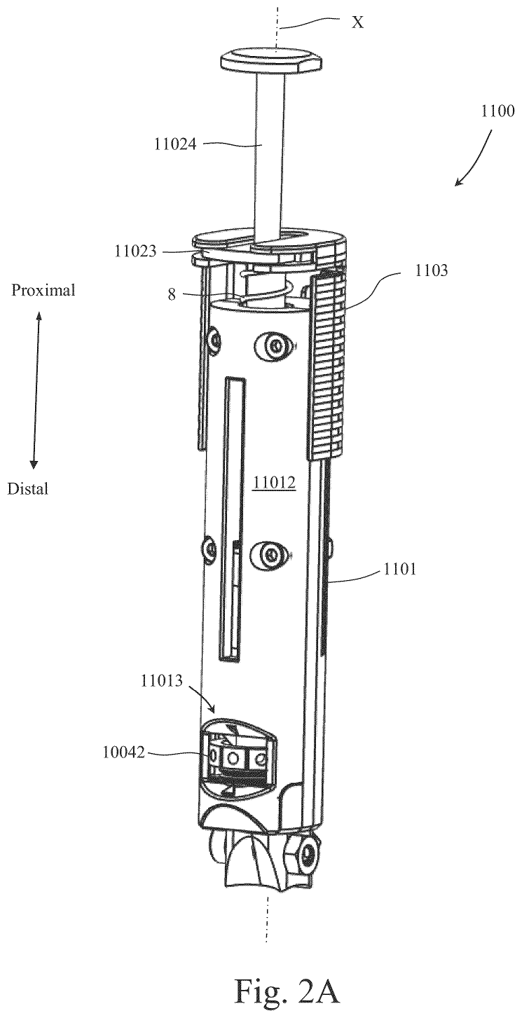

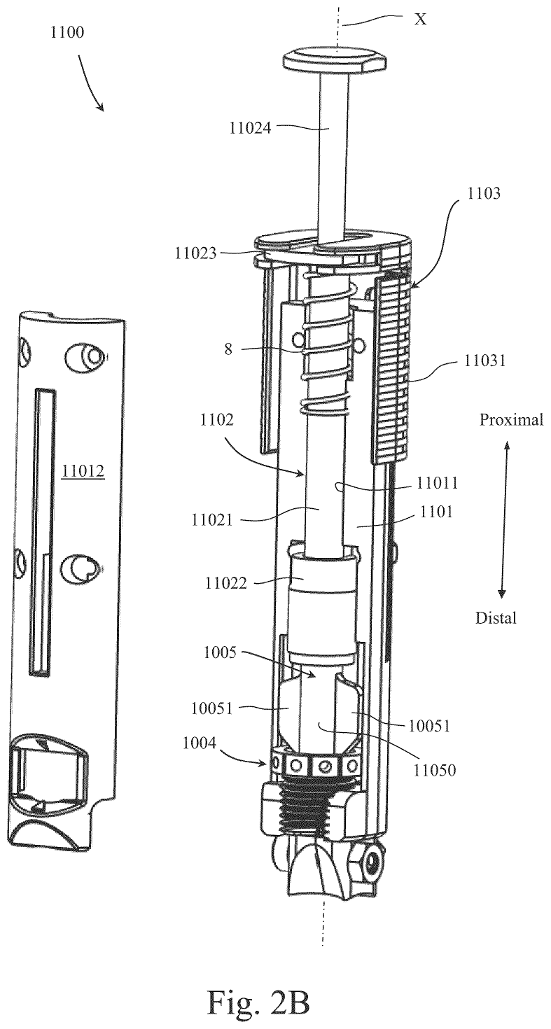

Attention is drawn to FIGS. 2A and 2B schematically showing respective non-exploded and partial exploded perspective side views of an embodiment of an injection device 1100 generally similar to the ones seen in FIGS. 1A and 1B.

Injection device 1100 is generally formed along a longitudinal axis X defining proximal and distal axial directions, and includes a main body 1101 generally formed about axis X. In FIG. 2B a side portion 11012 of the main body 1101 is shown removed—revealing a syringe member 1102 that is fitted at its barrel 11021 to the main body in a manner that permits the barrel to be axially slidable relative to the body through an axial passage 11011 that is formed within the body.

The syringe member in this example includes a widening 11022 at a relative distal side of the barrel and a flange 11023 at an upper proximal side of the barrel. The injection device further includes an actuator 1103 that is secured in this example at its proximal side to the barrel's flange with a biasing member (or spring) 8 of the device (here in form of a compression spring) being located between an inner proximal side of the actuator (here below the syringe's flange) and the syringe's body.

Actuator 1103 as seen in this example has a shroud 11031 that at least partially surrounds an upper proximal region of the device's main body 1101 and is adapted to axially slide along the outer side of the main body. A plunger 11024 of the syringe member projects in a proximal direction through the actuator 1103 away from the syringe's barrel.

The injection device further includes a needle member 1005 attached to a distal side of the syringe member. The needle member 1005 at its proximal side includes a hub 11050 with guiding means 10051 that are aimed at resisting rotation of the needle member about axis X as it is urged to slide along same axis X. In this example, the guiding means 10051 are formed as axially extending wing members that project radially outwards away from axis X and hub 11050.

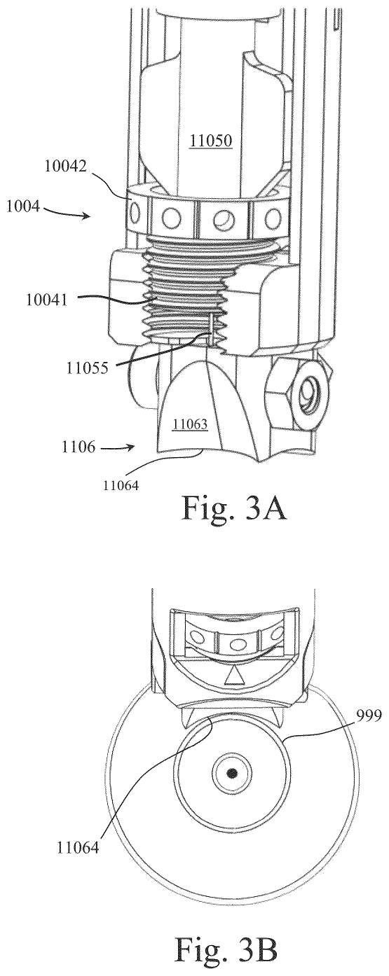

Injection device 1100 in FIG. 2B is also seen including an adjustable stopper 1104 at a distal lower side of main body 1101. With attention additionally drawn to FIG. 3A, stopper 1104 can be seen here being formed at its lower distal side with a thread 10041 that is threadingly engaged within a corresponding thread formed at a distal lower side the main body 1101. Rotation of the stopper in opposing directions about axis X is arranged to respectively lower or elevate the axial location of stopper within the lower side of the main body.

Stopper 1104 also includes at its upper side a toggle 10042 with markings, which as seen in FIG. 2A can be exposed to an exterior of the injection device here through a window 11013 formed at a relative lower distal side of the side portion 11012 of device's body. A physician or operator using the injection device may manually rotate the stopper via toggle 10042 in order to lower or elevate its location.

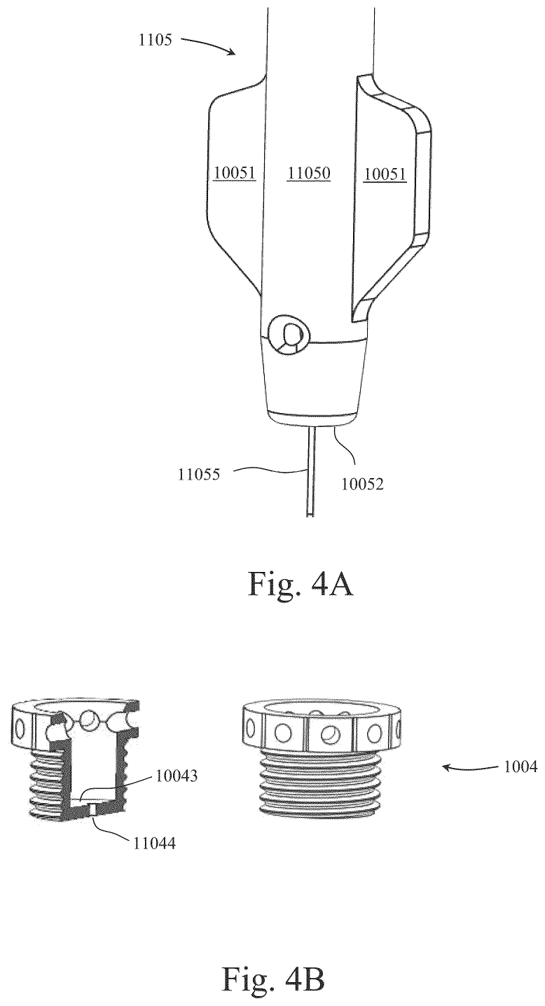

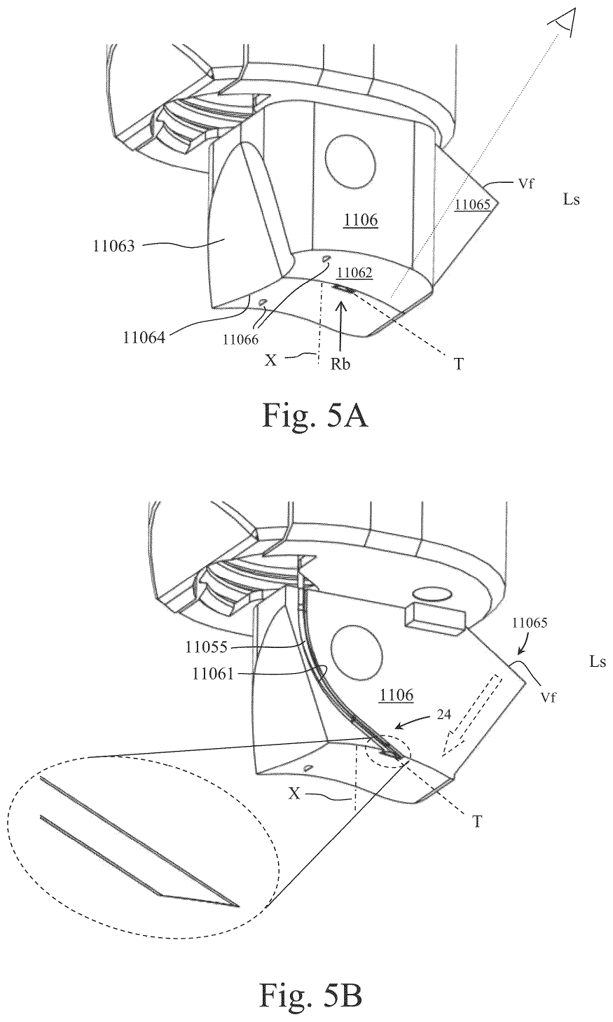

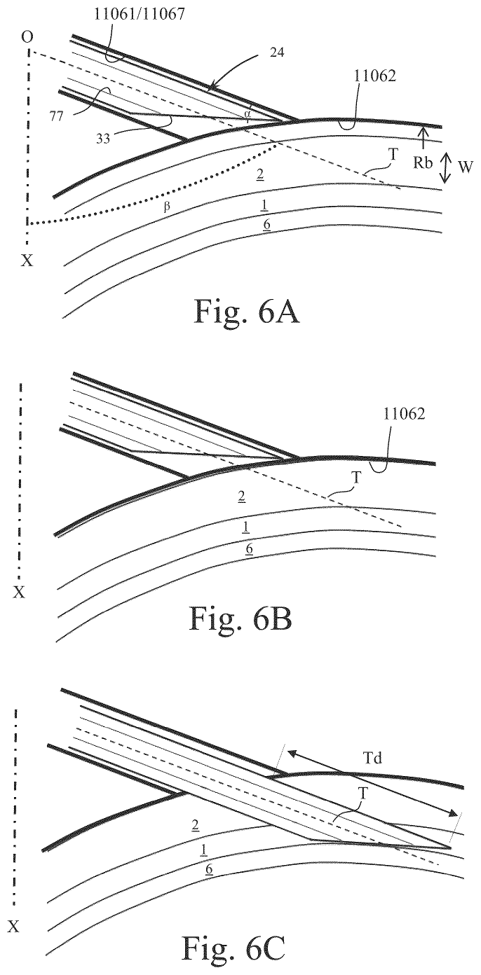

With attention still drawn to FIG. 3A, needle member 1005 can be seen projecting with its needle 11055 in a distal direction away from a distal end 10052 (see visible in FIG. 4A) of the needle member's hub 11050. The needle 11055 in its distal extension outside of the main body passes through a curved guiding channel 11061 (see visible in FIG. 5B) formed within an abutment member 1106 of the injection device, which is located at a distal lower side of the injection device.

The curved guiding channel 11061 is adapted towards its end to extend along a generally straight section to direct a terminal region 24 of the needle 11055 to extend (also on its route outside of the guiding channel) along an axis T that is transverse and/or inclined to the central axis X of the device. This extension along axis T may be along a vector direction that extends in combination generally in the axial distal direction and also towards a given one of the lateral sides ‘Ls’ of the device.

The needle 11055 may be threaded through a low friction tube (not shown) that is located within guiding channel 11061. The low friction tube (e.g. made from Teflon or the like) may facilitate smooth passage of the needle through the guiding channel as it is urged to moved and slide there through. The needle may be formed in one example from nitinol.

Attention is drawn to FIGS. 4A and 4B showing (see FIG. 4A) the needle 11055 that is fitted to the lower side of the syringe member. The stopper 1104 as seen in the cross section at the left-hand side of FIG. 4B has in this example a generally upward open cup shape formation for receiving therein from above a distal lower side of the needle member.

A floor 11043 with an aperture 11044 at its center is located in this example at a distal lower side of the stopper's cup shape formation—and the needle member is adapted when being urged to axially slide within the injection device, to distally move until it engages the stopper's floor 11043 with its distal end 10052, while the needle 11055 projects in the distal direction beyond the stopper via aperture 11044.

With attention drawn to FIGS. 5A and 5B, abutment member 1106 can be seen including an axial distal facing abutment face 11062. The abutment face 11062 may be formed to have a shape that generally corresponds to the outer shape of the body part or tissue that the injection device is intended to be used for injecting substances therein.

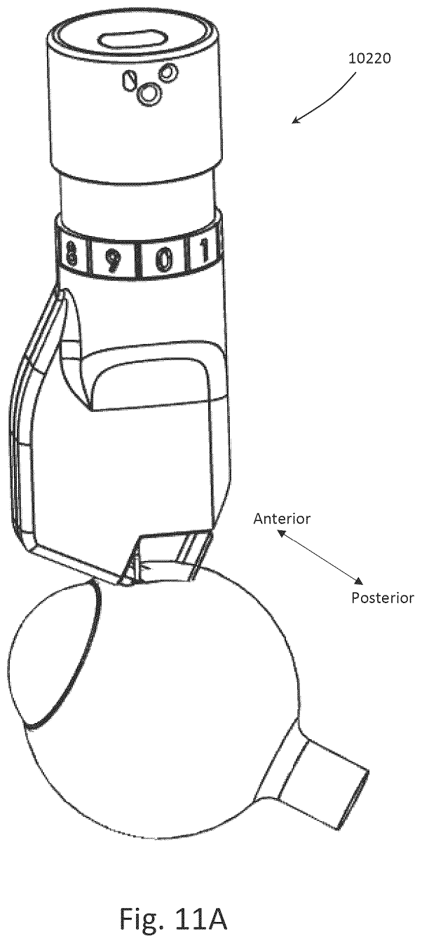

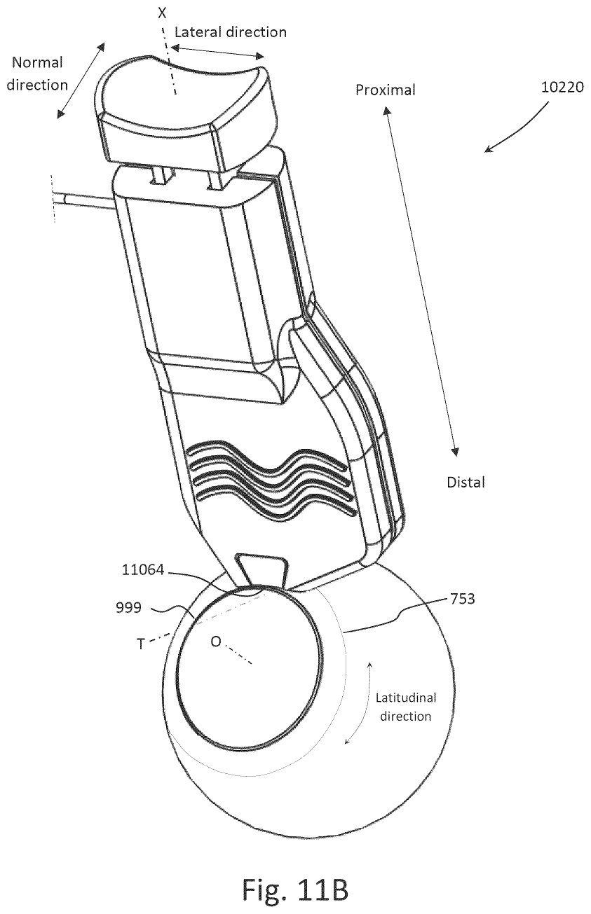

In the case where the injection device is intended for use e.g. for administrating medication substances into an eye tissue, the abutment face 11062 (at least in a cross section including or parallel to axes X and Y) may be designed to follow a general radius Rb by having e.g. a generally concave cylindrical or spherical or conical shape. The abutment member 1106 as seen may also be provided with a cut-away face 11063 formed at a lateral side of the abutment member, which may be as seen in this example at an opposite side to the given lateral side ‘Ls’ towards which axis T slants. A marking edge 11064 may be formed at an intersection between cut-away face 11063 and abutment face 11062. It is noted that marking edge 11064 (with or without such intersection with a cut away face) may also be formed at other side edges of the abutment member, such as at those located on opposing ‘normal’ sides of the abutment face (where the ‘normal’ direction being orthogonal to the ‘lateral’ direction). Such a marking edge at a normal side of the abutment member or face may be generally parallel to an imaginary surface including axes X and T in these views (see example provided in FIG. 11B and discussed herein below).

As seen in FIG. 3B, this marking edge 11064 may assist a physician in certain cases in positioning the injection device at a desired position with respect to observed body structures 999 in order to reach a targeted layer within a body tissue in proximity to such structures. In this example, where the targeted layer may be the choroid, which is located within the eye - such as an observed body structure 999 may be the limbus.

The abutment member may possibly further be formed with a viewing port 11065 that is generally located at the given lateral side ‘Ls’ of the device. The viewing port 11065 in embodiments where the abutment member is formed from transparent material may be formed from said same transparent material in order to provide to a physician using the injection device a glancing view (see ‘dashed’ arrow in FIG. 5B) towards the area where the tip of the needle is arranged to eject the medication substances.

An upper viewing face Vf of the viewing port may be designed to be generally parallel to axis T along which the terminal region 24 of the needle 11055 extends or moves. Viewing port 11065 may thus be aimed at reducing reflections that may obscure views of the needles tip, and in cases where viewing face Vf is generally parallel to axis T may assist in reducing the angle of incidence between the line of sight of the physician towards the needle's tip (see ‘dotted line’ in FIG. 5A) as it passes through the viewing face Vf.

Finally, the abutment member 1106 may be formed with one or more barb members 11066, preferably two or more of such barb members, which distally project beyond the abutment face 11062. The barb members 11066 may be formed at the opposing lateral side to lateral side ‘Ls’ in order to resist stretching/movement of the tissue through which the needle penetrates as it extends out of abutment face 11062 along axis T.

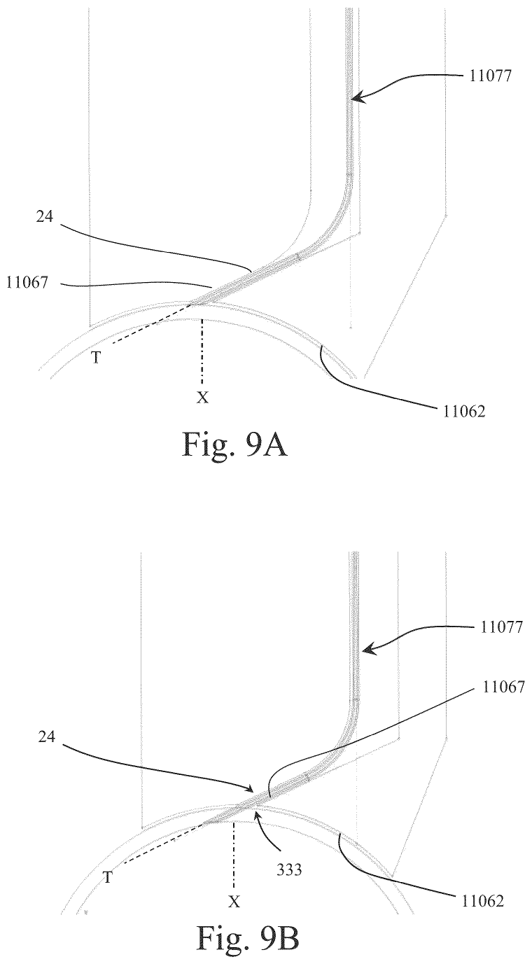

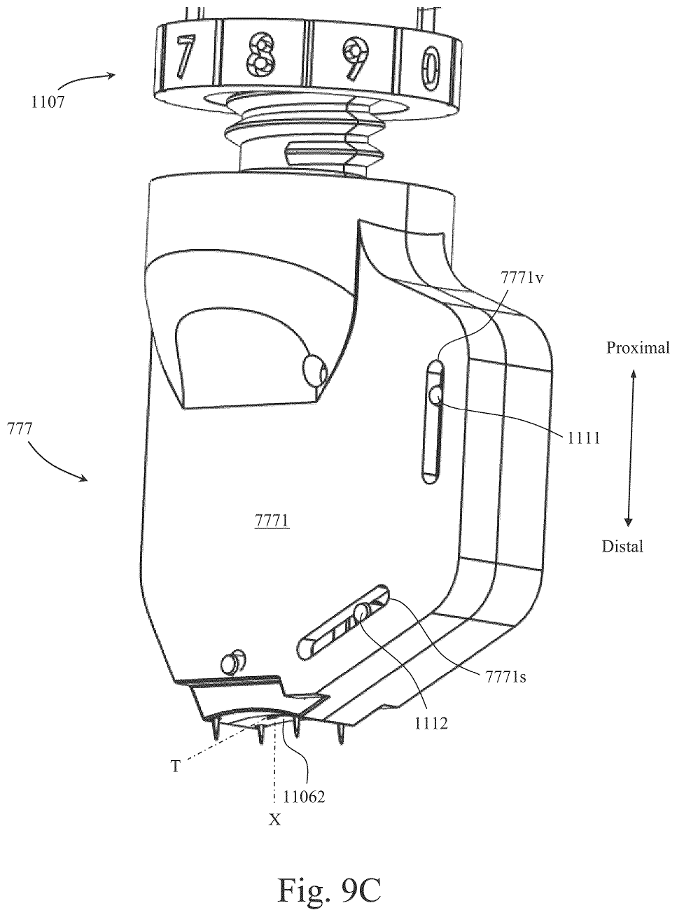

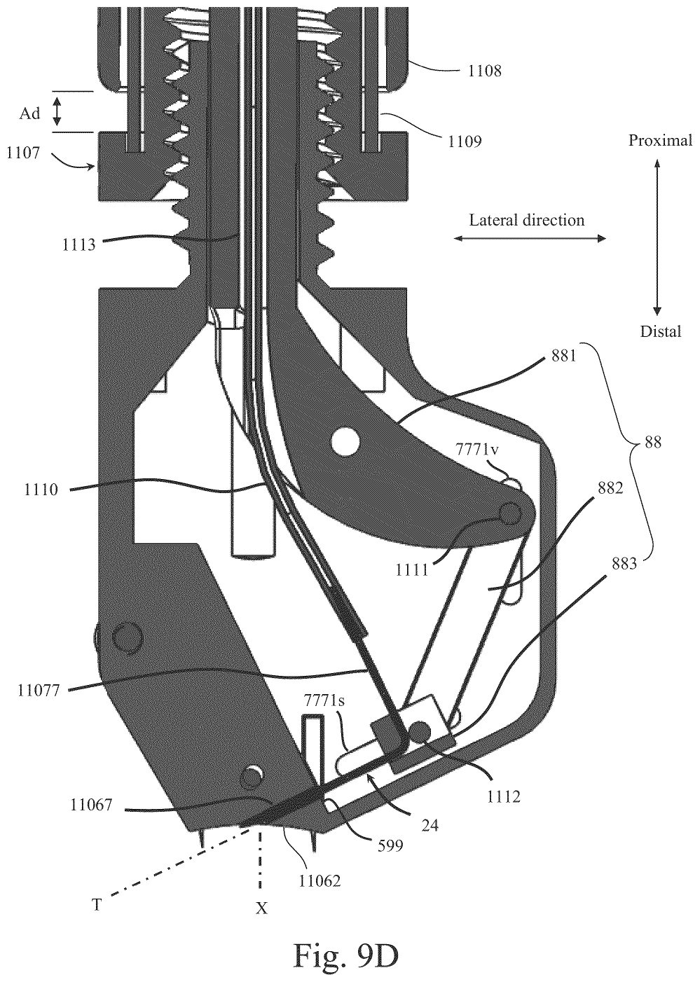

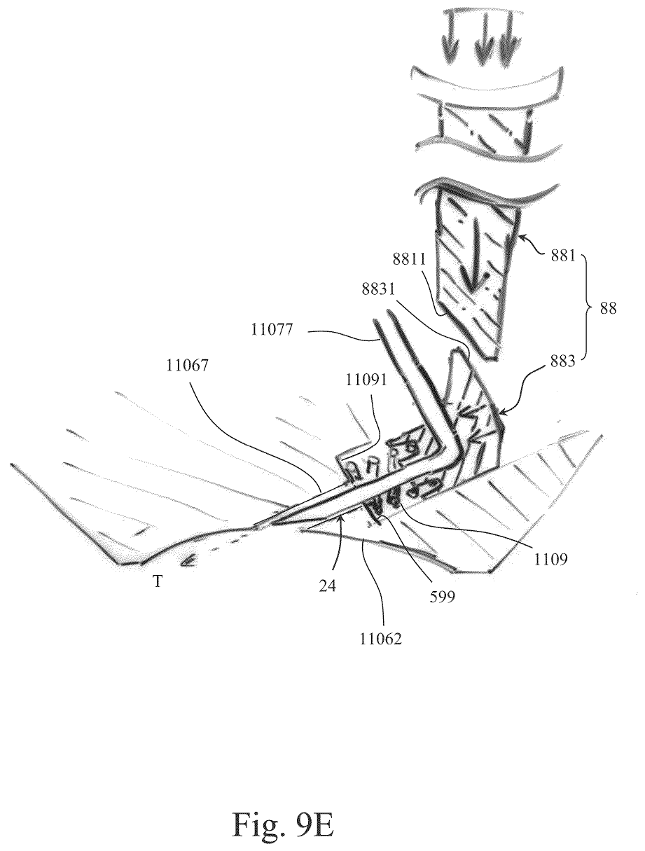

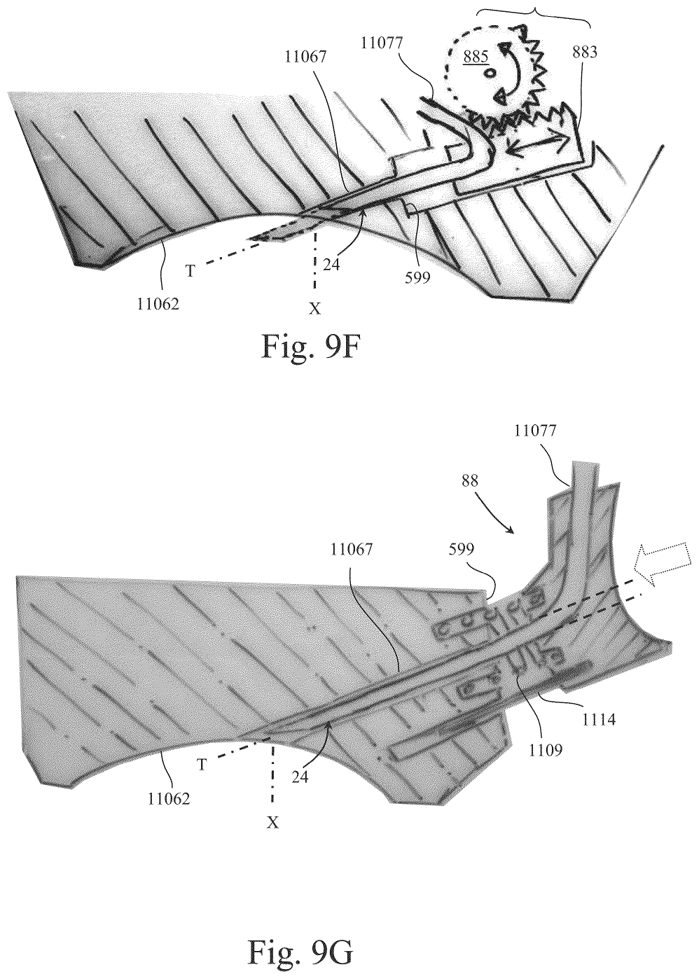

Attention is drawn to FIGS. 6A to 6C showing a terminal region 24 of a needle of an injection device in accordance with various embodiments of the present invention, during different stages of penetration into body tissue layers. This needle may be formed from various types of materials, such as from flexible material or relative rigid and inflexible material such as those discussed and shown e.g. in FIG. 9 onwards.

In FIG. 6A the injection device can be seen approaching a target area of a body tissue with its abutment face 11062 leading, while FIG. 6B shows the injection device already abutting and bearing against the target area of the body tissue with abutment face 11062. The biasing contact of abutment face 11062 that bears against the targeted body tissue—is arranged to urge the body tissue (and possibly tissue layers immediately below it) to conform to radius Rb of the abutment face 11062.

As indicated in FIG. 6A, axis T along which the terminal region 24 extends is tilted or inclined by an angle “beta” relative to the central axis X of the injection device. In other words, rotation of the injection device about axis X can be defined as forming with its axis T an imaginary coned surface that tapers upwardly (proximally) towards an apex O of the cone where axes X and T intersect, where said cone is defined as having a cone angle of “beta” between axes X and T. Although axis X is here seen illustrated at the left hand side of the intersection of axis T with abutment face 11062, it is noted that as seen in the various embodiments disclosed herein, axis X may be located also at other regions, such as generally passing through the intersection of axis T with the abutment face.

The needle can be seen including an internal channel 77 that opens out at a distal aperture 33 of the channel, and an internal bevel angle “alpha” of a chamfer formed at a tip of the needle is defined in a cross sectional plane that includes axis T and is perpendicular to the chamfer plane. Such angle “alpha” can be seen being formed as a generally small angle, e.g. smaller than about 45 degrees and preferably smaller than about 25 or possibly 15 degrees.

As seen in FIGS. 6A and 6B, the distal tip of the needle as it advances towards a targeted layer 1 for example of the choroid, initially exposes its sharped edged tip in this example towards the outer side in this example of the sclera 2 in a position/orientation suitable for penetrating into the sclera.

As seen in FIG. 6C, as the needle advances along axis T within the sclera 2 towards the targeted layer 1 of the choroid, the chamfer of the tip reaches targeted layer 1 (and/or a position within this ˜100 micron thin layer 1 in the example of it being the choroid) at a position where it is generally tangent to layer 1. In this position the needle is suited to release medication substances towards and/or into the targeted layer 1 with reduced likelihood of penetrating unintentionally through layers beyond targeted layer 1 such as the retina (or more precisely the RPE layer between the retina and choroid)-since its sharp edge is accordingly now less positioned in an ideal orientation to penetrate layer 1 (and in particular in this example retina 6). Technical measures such as the adjustable stopper or removable stopper and/or terminal stop (or the like), may assist in setting a limit for the needle to not advance too much, e.g. to a position where in may penetrate into the retina 6.

It is noted that various technical measures as disclosed herein, such as the optional guiding means 10051 or bend 87 or glue (or the like), which resist rotation of the needle member about its axis—may assist in ensuring that the tip of the needle is located initially with its sharped edged tip exposed towards the outer side of the body tissue in a position suitable to penetrate through it, and when reaching the targeted layer—in a position generally tangent to the targeted layer suitable for releasing medication substances towards this layer, while avoiding further penetration through the targeted layer.

Since the curvature of the body tissue to be treated may be generally dictated in this example by radius Rb of abutment face 11062 that bears against the body tissue—an additional parameter of the body tissue that may optionally assist in order to successfully reach a targeted layer within the body tissue, may be the thickness of penetration into the body to reach the targeted layer.

In an example where the eye is the body tissue to be treated as seen in FIGS. 6A to 6C—this may mean obtaining thickness W of the sclera 2 in order to assist in deriving a travel distance Td that the needle should preferably advance in its terminal region 24 along axis T beyond abutment face 11062—to position aperture 33 of the device's needle generally adjacent and/or within the targeted layer 1 e.g. of the choroid. Additional structural parameters that may assist in obtaining travel distance Td may include the device's angle “beta”.

Thickness W of penetration into the body tissue may be obtained in various techniques, such as by Optical Coherence Tomography (OCT) imaging, by a pachymeter device, by Ultrasound Biomicroscopy (UBM) technique (or the like).

Arriving at the travel distance Td may be obtained in various ways, such as by inserting the discussed parameters into the following equation.

$$Td = \frac{1}{2} ( - 2 W cos ( β ) - \sqrt{{( - 2 W cos ( β ) )}^{2} - 4 {Rb}^{2} - {( Rb - W )}^{2} )}$$

In the following discussion, the operation of the optional example of injection device 1100 will be described. In is noted that the other injection device embodiments disclosed herein may slightly differ in operation and/or in technical elements from injection device 1100, which is here provided as a mere example of one manner of operating of a certain optional embodiment of an injection device.

Injection device is designed in this optional example to be held by a physician with at least one hand engaging the device's actuator such as actuator 1103. The biasing member 8 that is located in-between main body 1101 and the actuator 1103—is arranged to urge the actuator together with the syringe member in an upward proximal direction until the syringe's widening 11022 reaches the narrower entry into passage 11011 to stop this upward motion. In this position, the injection device is maintained in a non-activated state ready for use.

An initial step of operating the injection device may be performed by first positioning the stopper of the device at a position defined by the travel distance Td that is expected in order to reach the targeted layer. This may be performed by manipulating the stopper's toggle according to the desired travel distance Td.

After this initial step, the device's abutment member 1106 may be urged to a position over a target location where injection is planned to take place. A physician operating the device may glance through viewing port 11065 to ensure that the injection device is suitably placed over the target site where injection of the medication substance is intended.

Once in place, activation of injection may start by urging the actuator in the distal direction in order to press the injection device towards the target site. The distal relative movement of the actuator against the biasing means 8 urges the barrel of the syringe member to move/slide together with it in the distal direction within main body until the distal end 10052 of the syringe member engages the floor of the adjustable stopper 1004. This in turn urges the needle to advance along axis T out and beyond the abutment face 11062 towards the target site within the body tissue.

Once this step has been accomplished and the needle's tip is in place, the physician-while holding the actuator pressed towards the patient—may urge the syringe's plunger in the distal direction relative to the barrel and by that urge the medication substance within the syringe's barrel to be emitted out of the needle's tip towards the target site.

By releasing the pressure applied upon the actuator, the physician may allow the actuator to be biased back upwards by the biasing means together with the barrel of the syringe member and the needle member that is attached to the syringe. This motion allows the needle to be retreated backwards until its tip reaches a position proximal to abutment face 11062 where it does not project in the distal direction beyond the abutment face 11062.

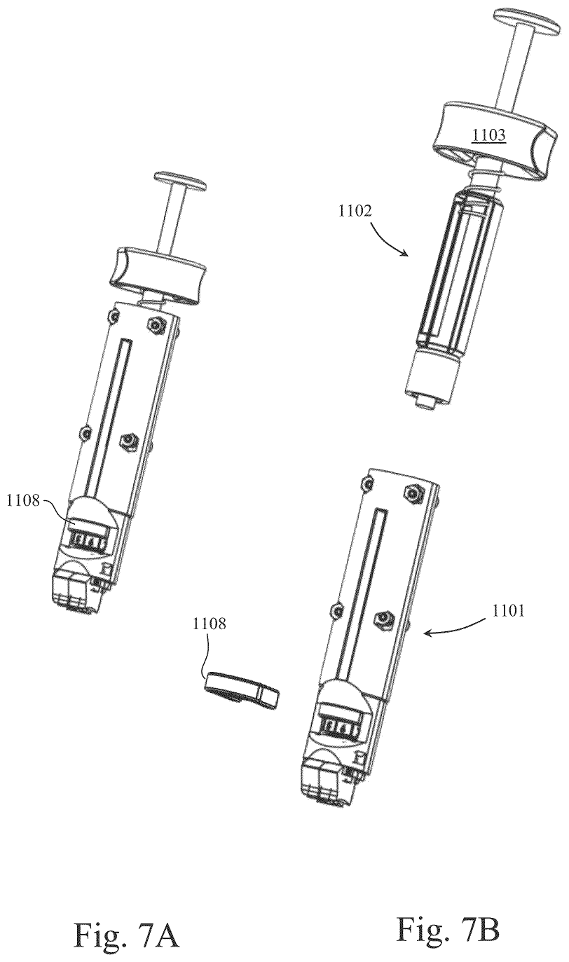

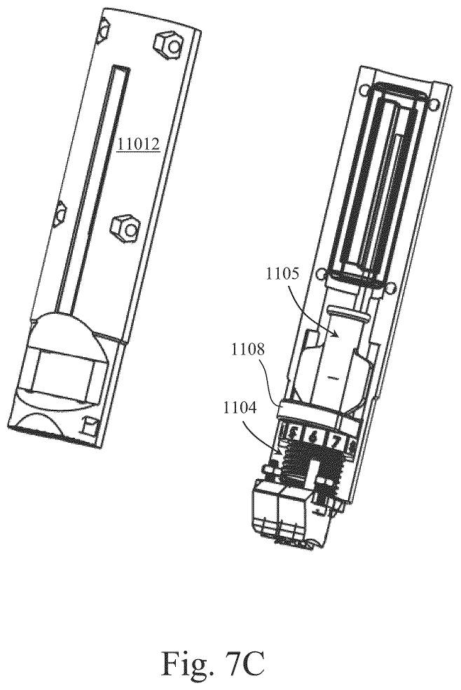

Attention is drawn to FIGS. 7A to 7C showing assembled (FIG. 7A) and partial exploded (FIGS. 7B and 7C) views of an injection device embodiment generally similar to the one shown in the former figures.

In this embodiment, the syringe member 1102 and actuator 1103 can be seen forming a sub-assembly that is detachable from the remainder of the device that here includes the main body 1101 of the device and the needle member 1105, stopper 1104 (and the like).

This injection device embodiment may facilitate drawing of medication substances into the syringe member when detached, and then fitting the syringe member into the remainder of the device to assume an assembled state of the device ready for use, where the syringe member is secured (e.g. via a lure lock or the like) within main body 1101 and fitted at its distal end to the needle member.

In this optional example a removable safety lock 1008 may be seen being placed between the needle member and the stopper 1104 while the syringe member is manipulated into its position within the assembled injection device—in order to avoid unintentional displacement of the needle member in the distal direction that may position it beyond the abutment member of the device during this assembly phase. Once the syringe member is in place, the safety lock 1008 may be removed when the device is to be used. It is noted that other mechanisms, some of which non-removable, may be equally feasible for functioning as a safety lock. For example, the safety lock may be embodied as a toggle (not shown) that may be toggled between an activated state where it resists unintentional displacement of the needle member in the distal direction and a non-activated state where displacement of the needle member in the distal direction can be performed when the actuator 1103 is pressed in the distal direction against a body tissue.

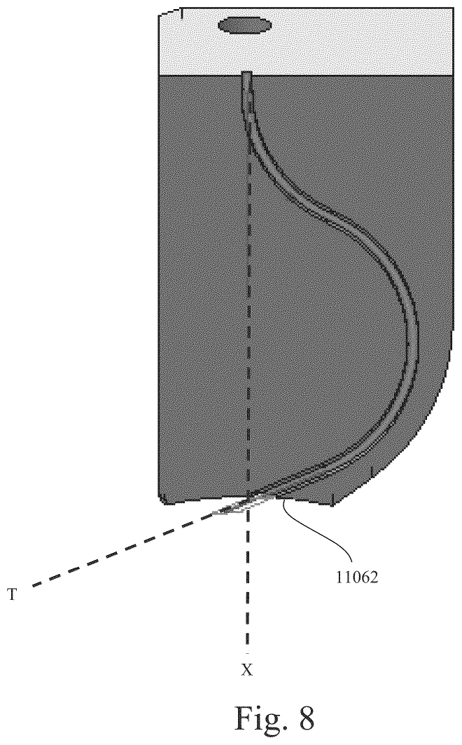

Attention is drawn to FIG. 8 showing a distal region of an injection device embodiment, illustrating an option where axes X and T may be designed to substantially intersect at the location where the needle is adapted to project out of the abutment member beyond its abutment face 11062. Such a design in certain cases may assist in stability of the device during initial penetration of the needle into a body tissue, due inter alia to axis X along which force is applied towards the body tissue being generally perpendicular to the abutment face at the point of needle penetration into the body tissue.

FIGS. 9A and 9B demonstrate a possible embodiment of an injection device where the needle 11077 may be formed from relative rigid and inflexible material such as stainless steel. In the embodiments described with respect to FIG. 9, such a relative rigid needle may be understood as meaning a hypodermic needle, which is commonly used in medical settings and is designed to penetrate the body tissue and/or underlying tissues.

As seen in this example, the needle 11077 may optionally be formed with a pre-designed curvature or bend at a distal region thereof defining a generally straight section forming a terminal region 24 of the needle leading to the needle's chamfered or beveled tip. Beveling of the needle's tip and use of the needle by the injection device embodiments seen in FIG. 9 may be generally similar to those exemplified and described with respect e.g. to FIGS. 6A to 6C. It is noted that the needle may alternatively be formed also as a substantially straight needle substantially devoid of any bends therein, while maintaining the desired orientation of the needle may be by fixing the needle to a sliding carriage by other means such as glue (or the like).

In such a design, urging the needle's terminal region 24 to penetrate via an opening 333 at the abutment face beyond the injection device's abutment face 11062 along axis T may be seen being implemented by providing a generally straight needle guide 11067, here exemplified as an angled generally straight tubular needle guide defining axis T, through which the needle passes as it is urged beyond the device's abutment face. Tubular guide may be a separate guide member placed within the device's distal frame/region for guiding the needle along axis T or may be formed as an integral part of the device.

Needle guide 11067 functions as a rail that guides movement of the needle's terminal region 24 along the distal inclined direction of axis T (as seen e.g. in FIGS. 6A to 6C) to a position suitable for injecting medications towards a target layer within a body tissue.

Attention is additionally drawn to FIGS. 9C to 9E showing further details of an aspect of the present invention initially seen in FIGS. 9A and 9B—relating to the possible use of a relative rigid and inflexible needle such as 11077 in the various injection device embodiments disclosed herein.

In FIG. 9C, a distal terminal region of an injection device is shown having an outer housing 777 in this example including generally parallel housing faces 7771 (here only one of the housing faces, which faces out of the page is visible). The angled generally straight tubular guide 11067 (see FIG. 9D), possibly formed as an at least partially covered groove or channel, may be an integral part of housing 777 or a separate part embedded within housing 777. A lower side of the outer housing is seen including in this example the abutment face 11062 of the injection device from which optionally several (here possibly four) barb members may project in the distal direction. An adjustable stopper 1107 of the injection device is here seen being located proximally above the housing 777.

Attention is drawn to FIG. 9D showing a cross sectional view of the terminal region of the injection device seen e.g. in FIG. 9C revealing the interior of housing 777. In this view, the injection device can be seen including an actuating mechanism 88 that includes a pusher 881, a link rod 882 and a sliding carriage 883. Also seen in this view is the stopper 1107 and a shroud 1108 (such as shroud 11031 seen in FIG. 2B) that may be part of the actuating mechanism 88.

The axial distance Ad between the stopper and the shroud's distal lower end defines the travel distance Td (see FIG. 6C) that the needle can advance in its terminal region 24 along axis T beyond the device's abutment face 11062. Axial distance Ad in the various embodiments disclosed herein may be generally the same or different to travel distance Td depending on the mechanism used for actuating movement of the needle. A biasing member (or spring) 1109 pressed between the stopper and an upper section (not seen) of the actuating mechanism may be configured to retract needle back so it does not protrude beyond the abutment face when no force is applied downwards upon the upper section actuating mechanism e.g. by a physician.

The outer housing 777 in this example as seen in FIG. 9C includes two optional generally axially extending parallel vertical slots 7771v each formed in a respective one of the housing faces 7771 and two optional generally parallel slanted slots 7771s each formed in a respective one of the housing faces 7771. The slanted slots 7771s extend generally parallel to or along axis T and to needle guide 11067.

A minimal volume connecting tubing 1110, possibly made from silicon, Teflon, thermoplastic PVC (or the like)—can be seen extending in this example optionally through a lumen 1113 formed in pusher 881 to connect at its lower side to an upstream proximal side of needle 11077 and at its upper side to a syringe member (not shown) of or associated with the injection device. The syringe in one optional example may be located at a proximal side of the pusher possibly encased within the shroud 1108 to be moved together with the pusher as it is pressed to urge the needle to protrude beyond the device's abutment face. In other examples, the syringe may be located in other locations.

A first pin 1111 threaded through a distal lower end of the pusher 881 and through the vertical slots 7771v constrains movement of the lower end of the pusher 881 along the vertical slots 7771v and a second pin 1112 threaded through the sliding carriage 883 and through the slanted slots 7771s constrains movement of the sliding carriage 883 along the slanted slots 7771s. The first pin 1111 is also threaded through a first end of the link rod 882 and the second pin 1112 is also threaded through a second opposing end of the link rod 882 in order to link between movements of the push arm 881 and sliding carriage 883.

As seen, in this example the distal lower end of the pusher 881 where first pin 1111 connects the pusher 881 to link rod 882, protrudes slightly in a first lateral direction of the device in order to orient link rod 882 such that it slants downwards and in an opposing lateral direction towards the location where second pin 1112 couples it to sliding carriage 883. Such positioning of first pin 1111 above the second pin 1112 and to its lateral right, together here with the resulting downward slanting link rod 882, assists in promoting movement of the sliding carriage 883 along a direction generally parallel to axis T.

The needle 11077 is fitted here at or adjacent its bend to the sliding carriage 883 so that movement of the sliding carriage 883 generally along an axis T defined by the needle guide 11067—is adapted to move the terminal region 24 of the needle along same axis T.

The needle is fitted to the sliding carriage here with assistance of the bend to maintain the chamfer/bevel formed at the needle's tip in an orientation where it faces distally away from the abutment face of the injection device towards a body tissue it aims to penetrate as seen e.g. in FIGS. 6A to 6C. It is noted that the needle may alternatively be formed also as a substantially straight needle substantially devoid of any bends therein, while maintaining the desired orientation of the needle may be by fixing the needle to the sliding carriage by other means such as glue (or the like).

As seen in the cross section of FIG. 9D, the needle guide 11067 opens inwards towards the sliding carriage 883 via a terminal stop 599, and this terminal stop 599 acts as a terminal position up to which the sliding carriage 883 can move and advance needle 11077 to protrude beyond the device's abutment face.

Axially urging the pusher 881 in the distal direction is adapted to urge the needle's terminal region 24 via the sliding carriage 883 to advance along axis T to position the needle's distal tip beyond the injection device's abutment face. Retracting the pusher 881 back up in the proximal direction is adapted to urge the needle's terminal region 24 via the sliding carriage 883 to retract back along axis T to position the needle's distal tip at a position where it does not protrude beyond the injection device's abutment face.

Attention is drawn to FIG. 9E showing a distal region of an embodiment of an injection device having a relative rigid needle 11077. Here the actuating mechanism 88 can be seen including a pusher 881 and a sliding carriage 883. Pressing the pusher 881 in the distal direction urges a slanted distal face 8811 of the pusher to engage a slanted rear face 8831 of the sliding carriage 883.

This results in movement of the sliding carriage 883 generally along axis T, which is defined by the generally straight needle guide 11067 and in turn in movement of the needle 11077 that is fixed to the carriage along axis T to position its tip at a location beyond the abutment face 11062. The angled generally straight tubular guide 11067, possibly formed as an at least partially covered groove or channel, may be an integral part of the distal region or a separate part embedded within the distal region of the device. A biasing member 1109 is here seen being pressed between a forward side of the sliding carriage 883 and a bearing face 11091 within the device's distal region.

As seen in the cross section of FIG. 9E, the needle guide 11067 opens inwards towards the sliding carriage 883 via a terminal stop 599, and this terminal stop 599 acts as a terminal position up to which the sliding carriage 883 can move and advance needle 11077 to protrude beyond the device's abutment face

When pressure applied upon the pusher 881 is released, the biasing member 1109 is adapted to urge sliding carriage 883 back and by that retract also the needle back to a position where its tip does not protrude beyond the abutment face. The retracting sliding carriage 883 is adapted to also urge pusher 881 back up through interaction between the faces 8831, 8811.

Although not shown, a minimal volume connecting tubing such as 1110 is also included in this injection device embodiment for channeling substances during activation of the device from a syringe (also not shown) through needle 11077 towards a targeted layer within a body tissue.

Attention is drawn to FIG. 9F showing yet another distal region of an embodiment of an injection device having a relative rigid needle 11077. Here the actuating mechanism 88 can be seen including a pinion 885 with outer teeth on its periphery that mesh with corresponding teeth formed on the sliding carriage 883.

Rotating the pinion in a first rotational direction is adapted to urge the sliding carriage 883 generally along axis T, which is defined by a generally straight guide needle 11067 through which the needle passes. The angled generally straight tubular guide 11067, possibly formed as an at least partially covered groove or channel, may be an integral part of the distal region or a separate part embedded within the distal region of the device. The needle 11077, which is fixed to the carriage moves together with it along axis T to position its tip at a location beyond the abutment face 11062.

As seen in the cross section of FIG. 9F, the generally straight needle guide 11067 opens inwards towards the sliding carriage 883 via a terminal stop 599, and this terminal stop 599 acts as a terminal position up to which the sliding carriage 883 can move and advance needle 11077 to protrude beyond the device's abutment face

Rotating the pinion in a counter second rotational direction is adapted to urge the sliding carriage 883 back and by that retract also the needle back to a position where its tip does not protrude beyond the abutment face. The rotation of the pinion in the counter second rotational direction may be urged by a spring (not shown), possibly a torsion spring wrapped in a coil about the pinion's axis.

Although not shown, a minimal volume connecting tubing such as 1110 is also included in this injection device embodiment for channeling medication substances during activation of the device from a syringe (also not shown) through needle 11077 towards a targeted layer within a body tissue.

Attention is drawn to FIG. 9G showing yet a further distal region of an embodiment of an injection device having a relative rigid and inflexible needle 11077. Here the actuating mechanism 88 is embodied as a button like sliding carriage member that can be manually pressed as indicated by the ‘dotted’ arrow to move together with the needle that is fitted thereto along axis T in order to position the needle's tip beyond the abutment face 11062.

As seen in the cross section of FIG. 9G, the generally straight needle guide 11067 opens inwards towards the actuating mechanism 88 via a terminal stop 599, and this terminal stop 599 acts as a terminal position up to which the actuating mechanism 88 can move and advance needle 11077 to protrude beyond the device's abutment face

A biasing member 1109 pressed between a forward side of the sliding carriage and a bearing face within the device's distal region, is adapted when pressure applied upon the sliding carriage is released, to urge sliding carriage back together with the needle that is attached thereto—in order to retract it back to a position where its tip does not protrude beyond the abutment face.

As seen by the ‘dashed’ lines added in FIG. 9G, the needle may be formed also as a substantially straight needle devoid of any bends therein that may otherwise be used for assisting in maintaining the needle's desired suitable orientation positioning its chamfer/bevel facing generally downwards (see e.g. FIG. 6C).

Instead, for maintaining the desired orientation, the needle may be fixed to the sliding carriage by other means such as glue (or the like), and in this example a guide 1114 that is possibly fixed at one end e.g. to the sliding carriage and at its other end may be arranged to slide through a bore formed in the device's distal region-may assist in maintaining the suitable preferable orientation of the needle's chamfer during movement of the sliding carriage.

Although not shown, a minimal volume connecting tubing such as 1110 is also included in this injection device embodiment for channeling medication substances during activation of the device from a syringe (also not shown) through needle 11077 towards a targeted layer within a body tissue.

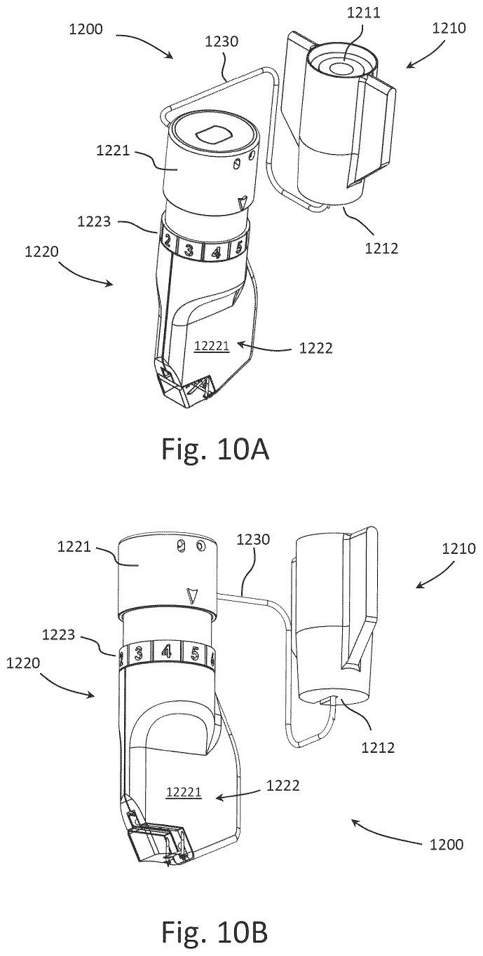

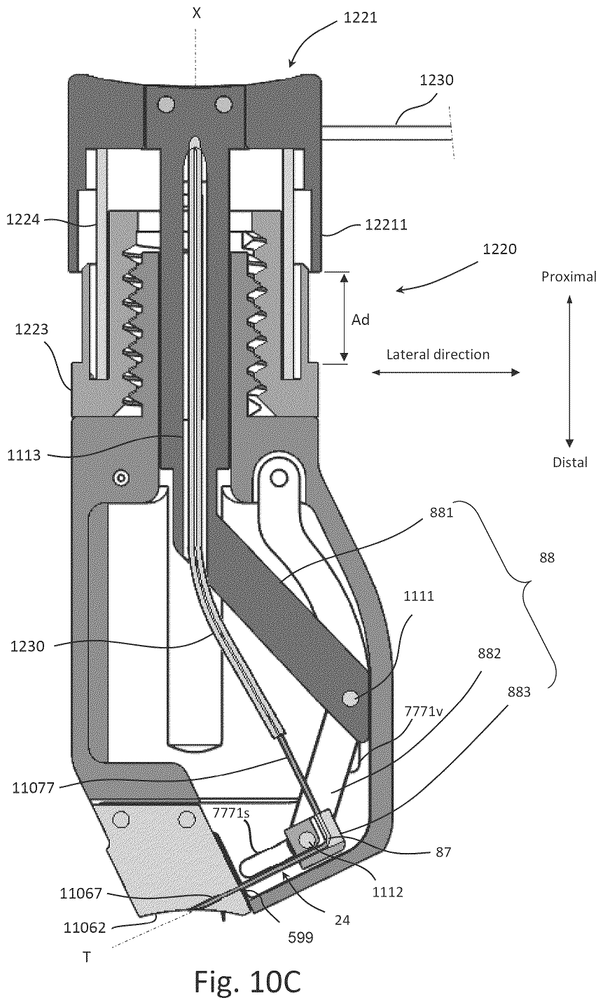

Attention is drawn to FIGS. 10A to 10C showing an embodiment of an injection device 1200 generally similar to those in former embodiments (such as those seen in FIGS. 9C and 9D), however here being designed to be formed as two main separate members, one being a syringe hub member 1210 and the other being an injector member 1220, which are fluidly connected by a flexible minimal volume connecting tubing 1230.

The injector member 1220 may include a needle 11077 (see FIG. 10C) that may be formed from relative rigid and inflexible material such as stainless steel. Such a relative rigid needle may be understood as meaning a hypodermic needle, which is commonly used in medical settings and is designed to penetrate the body tissue and/or underlying tissues.

The syringe hub member 1210 includes a receiving side 1211 to which a Luer of a syringe (not seen) may be attached and the tubing 1230 connects in this example to a different, here opposing, emitting side 1212 of the syringe hub member, in order to communicate medication substance emitted by a syringe towards the injector member 1220.

A distal terminal region of injector member 1220 has an outer housing 1222 in this example including generally parallel housing faces 12221 (in FIGS. 10A and 10B only one of the housing faces, which faces out of the page is visible).

Attention is additionally drawn to the cross section of FIG. 10C. As best seen in this cross section, the injector member 1220 includes an actuator 1221 having a peripheral distally extending shroud 12211. An angled generally straight tubular guide 11067 seen in this cross section, possibly formed as an at least partially covered groove or channel, may be an integral part of housing 1222 or a separate part embedded within housing 1222.

A lower side of the outer housing is also seen including in this example a concave the abutment face 11062 of the injector member 1220 from which optionally several (here possibly two) barb members may project in the distal direction.

In this embodiment, the injector member 1220 can be seen including an actuating mechanism 88 generally similar e.g. to the one seen and described in FIGS. 9C and 9D.

The actuating mechanism 88 accordingly includes a pusher 881, a link rod 882 and a sliding carriage 883. Also seen in this view is the adjustable stopper 1223 against which the shroud 12211 of the actuator 1221 is adapted to engage when being pressed axially distally downwards.

The axial distance Ad between the stopper 1223 and the shroud's 12211 distal lower end defines the travel distance Td (see FIG. 6C) that the needle can advance in its terminal region 24 along axis T beyond the device's abutment face 11062.

Axial distance Ad in the various embodiments disclosed herein may be generally the same or different to travel distance Td depending on the mechanism used for actuating movement of the needle.

A spring 1224 pressed between the adjustable stopper and an upper section of the actuator may be configured to retract the device's needle back so it does not substantially protrude beyond the abutment face when no force is applied downwards upon the actuator e.g. by a physician.

The outer housing 1222 in this example includes two optional generally axially extending parallel vertical slots 7771v each formed in a respective one of the housing faces 12221 and two optional generally parallel slanted slots 7771s each formed in a respective one of the housing faces 12221.

In the cross section of FIG. 10C only one vertical slot 7771v and one slanted slot 7771s is visible. The slanted slots 7771s extend generally parallel to or along axis T, which is defined by the generally straight needle guide 11067.

The minimal volume connecting tubing 1230, possibly made from silicon, Teflon, thermoplastic PVC (or the like)—can be seen extending in this example optionally through a lumen 1113 formed in pusher 881 to connect at its lower side to an upstream proximal side of needle 11077.

A first pin 1111 threaded through a distal lower end of the pusher 881 and through the vertical slots 7771v restricts or constrains movement of the lower end of the pusher 881 along the vertical slots 7771v and a second pin 1112 threaded through the sliding carriage 883 and through the slanted slots 7771s restricts or constrains movement of the sliding carriage 883 along the slanted slots 7771s.

The first pin 1111 is also threaded through a first end of the link rod 882 and the second pin 1112 is also threaded through a second opposing more distal end of the link rod 882 in order to link between movements of the push arm 881 and the sliding carriage 883.

As seen, in this example the distal lower end of the pusher 881 where first pin 1111 connects the pusher 881 to link rod 882, protrudes slightly in a first lateral direction of the device in order to orient link rod 882 such that it slants downwards and in an opposing lateral direction towards the location where second pin 1112 couples it to sliding carriage 883. Such positioning of first pin 1111 above the second pin 1112 and to its lateral right hand side, together here with the resulting downward slanting link rod 882, assists in promoting movement of the sliding carriage 883 along a direction generally parallel to axis T.

The needle 11077 is fitted here at or adjacent its bend 87 to the sliding carriage 883 so that movement of the sliding carriage 883 generally along an axis T defined by the generally straight needle guide 11067—is adapted to move the terminal region 24 of the needle along same axis T.

The needle is fitted to the sliding carriage here with assistance of the bend to maintain the chamfer/bevel formed at the needle's tip in an orientation where it faces distally away from the abutment face of the injection device towards a body tissue it aims to penetrate as seen e.g. in FIGS. 6A to 6C. It is noted that the needle may alternatively be formed also as a substantially straight needle substantially devoid of any bends therein, while maintaining the desired orientation of the needle may be by fixing the needle to the sliding carriage by other means such as glue (or the like).

As seen in the cross section of FIG. 10C, the generally straight needle guide 11067 opens inwards towards the sliding carriage 883 via a terminal stop 599, and this terminal stop 599 acts as a terminal position up to which the sliding carriage 883 can move and advance needle 11077 to protrude beyond the device's abutment face.

Axially urging the pusher 881 in the distal direction is adapted to urge the needle's terminal region 24 via the sliding carriage 883 to advance along axis T to position the needle's distal tip beyond the injection device's abutment face. Retracting the pusher 881 back up in the proximal direction is adapted to urge the needle's terminal region 24 via the sliding carriage 883 to retract back along axis T to position the needle's distal tip at a position where it does not substantially protrude beyond the injection device's abutment face.

Attention is drawn to FIG. 11A showing use of an injection device, such as the ones seen in FIGS. 10, 12 to 14 and 16 with only its injector member 10220 being shown. In is noted however that the principles seen and discussed with respect to these exemplary embodiments relating to the method of injecting substances—are relevant also to the other injection device embodiments disclosed herein.

Here the injector member 10220 is positioned to direct its needle when protruding beyond its abutment face, to extend along axis T in a posterior direction of the eye in order to direct medication substances e.g. generally towards the eye's macula.

With attention drawn to FIG. 11B, an injection device, such as the ones in FIGS. 10, 12 to 14 and 16 is seen with only its injector member 10220 being visible. In is noted however that the principles seen and discussed with respect to these exemplary embodiments relating to the method of injecting medication substances—are relevant also to the other injection device embodiments disclosed herein.

In this example, the injection device can be seen having a marking edge 11064 (such as the one seen and discussed with respect e.g. to FIG. 3B), however here being formed at opposing normal sides of the device's abutment member (where the ‘normal’ direction being orthogonal to the ‘lateral’ direction). In this example therefore, the marking edges 11064 (only one being visible) are generally parallel to an imaginary plane including axes X and T of the injection device.

In this view, the injection device is oriented relative to the eye by abutting the eyeball's outer side with its abutment face and placing one of its marking edges 11064 generally tangent to the eye's limbal ring (which here serves as an observed body structure 999).

Also indicated in FIG. 11B is the eye's optic axis O and an imaginary circular ‘latitudinal’ line 753, which circles optical axis O upon the eyeball's outer side. This ‘latitudinal’ line 753 lies on an imaginary plane that is orthogonal to the eye's optic axis O, and defines a ‘latitudinal’ direction about optic axis O upon the eyeball's outer side.

In this example, the ‘latitudinal’ line 753 being indicated generally passes through the opening in the device's adjustment face through which the device's needle is designed to protrude towards its targeted layer within the eye along axis T.

Along such a ‘latitudinal’ line the width of the eye's sclera is typically generally similar. As a result, orienting the device to protrude its needle into the eye generally tangent to such a ‘latitudinal’ direction provides a higher degree of certainty for a physician operating the injection device as to the depth to which the device's needle needs to penetrate into the eye in order to reach with its tip a targeted layer within the eye, such as the choroid that is beneath the sclera. Introducing a needle into this region of the eye adjacent the limbal ring as seen (e.g. about 3 mm from the limbal ring) is also a relatively safer target for various ophthalmic procedures.

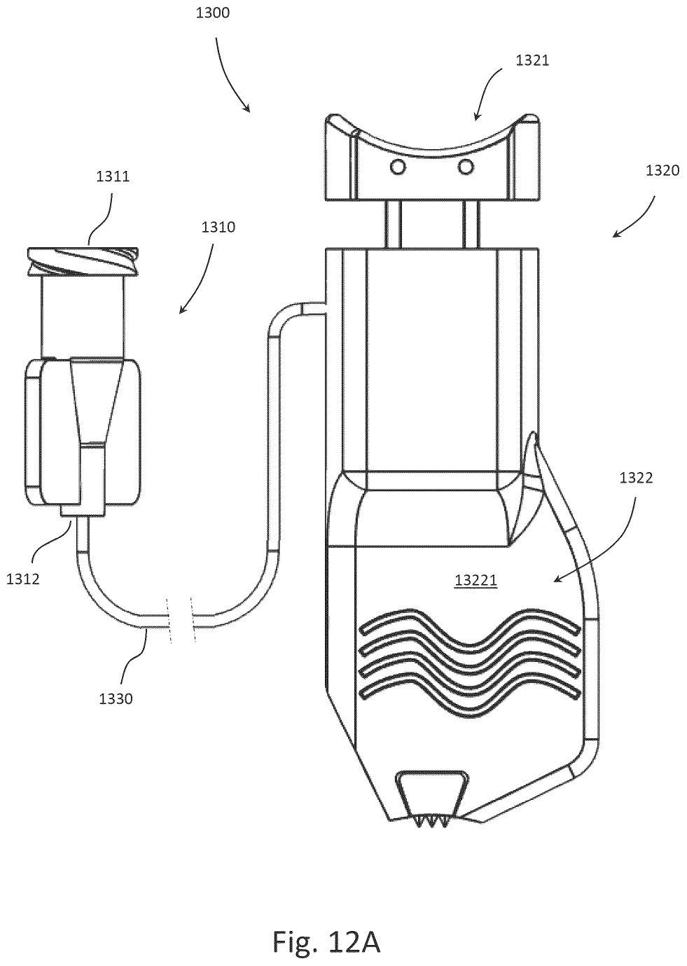

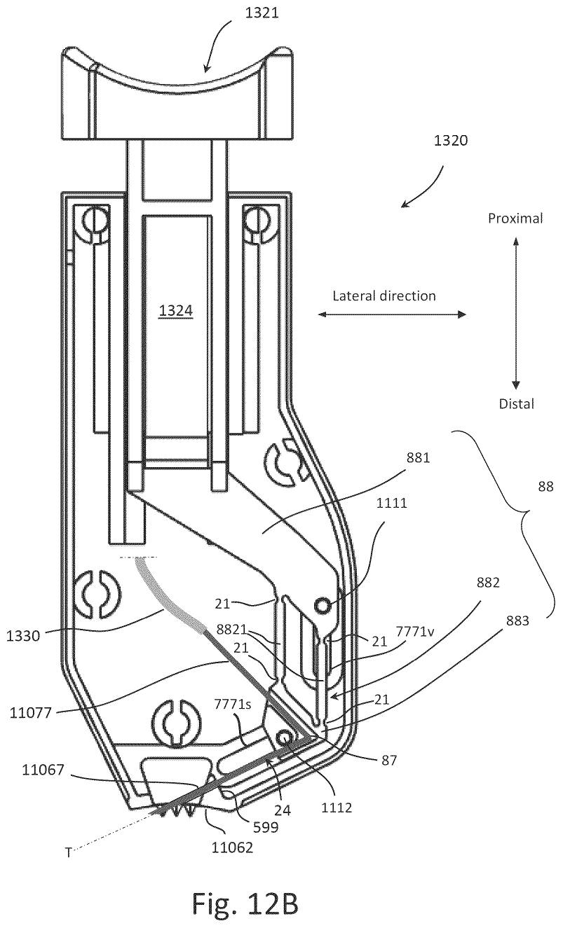

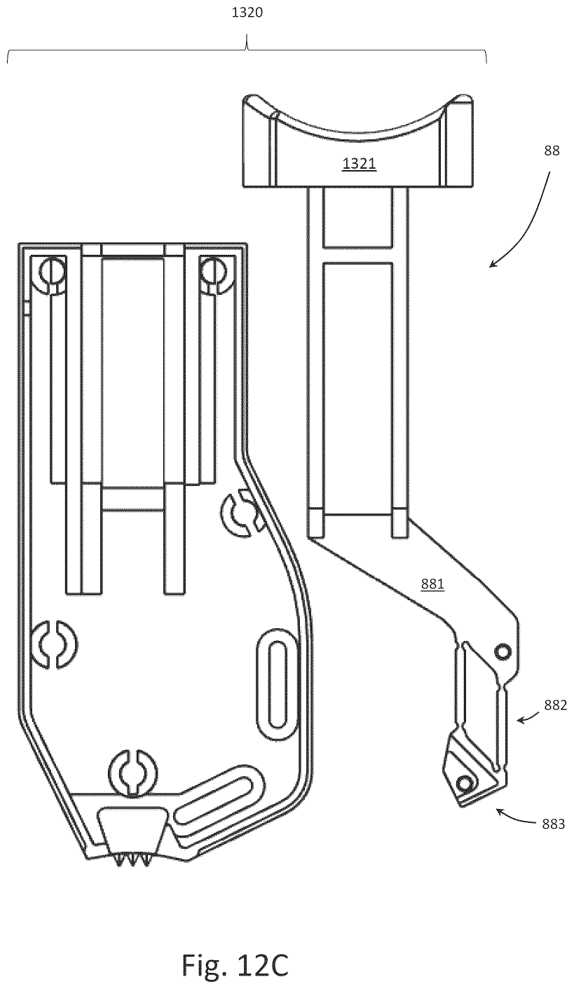

Attention is drawn to FIGS. 12A to 12C showing an embodiment of an injection device 1300 generally similar to the one seen and discussed with respect to FIGS. 10A to 10C, which includes two main separate members, one being a syringe hub member 1310 and the other being an injector member 1320, which are fluidly connected by a flexible minimal volume connecting tubing 1330.

The injector member 1320 may include a needle 11077 that may be formed from relative rigid and inflexible material such as stainless steel. Such a relative rigid needle may be understood as meaning a hypodermic needle, which is commonly used in medical settings and is designed to penetrate the body tissue and/or underlying tissues.

The syringe hub member 1310 accordingly includes a receiving side 1311 to which a Luer of a syringe (not seen) may be attached and the tubing 1330 connects in this example to a different, here opposing, emitting side 1312 of the syringe hub member to communicate medication substance emitted by such a syringe towards the injector member 1320.

A distal terminal region of injector member 1320 has an outer housing 1322 in this example including generally parallel housing faces 13221 (in FIG. 12A only one of the housing faces, which faces out of the page is visible).

As best seen in the cross section of FIG. 12B, the injector member 1320 includes an actuator 1321 and a generally straight angled tubular guide 11067 possibly formed as an at least partially covered groove or channel, which may be an integral part of housing 1322 or a separate part embedded within housing 1322.

A lower side of the outer housing is also seen including in this example a concave abutment face 11062 of the injector member 1320 from which optionally several barb members may project in the distal direction.

In this embodiment, the injector member 1320 can be seen including an actuating mechanism 88. While actuating mechanism 88 may be generally similar e.g. to the one seen and described in FIG. 10C, in this example the actuating mechanism 88 may be seen embodied having an integral formation in which its actuator 1321, pusher 881, link rod 882 and a sliding carriage 883 are integrally formed as a unitary one piece structure.

As seen in the cross section of FIG. 12B, the generally straight needle guide 11067 opens inwards towards the sliding carriage 883 via a terminal stop 599, and this terminal stop 599 acts as a terminal position up to which the sliding carriage 883 can move and advance needle 11077 to protrude beyond the device's abutment face

With attention additionally drawn to the exploded cross sectional view of FIG. 12C, the unitary one piece integrally formed structure of the actuating mechanism 88 of injector member 1320 can be seen including actuator 1321, pusher 881, link rod 882 and sliding carriage 883.

In the embodiment of FIGS. 12A to 12C it is seen that the injector member may be designed to be devoid of an adjustable stopper (such as adjustable stopper 1223 seen in FIG. 10) for limiting movement of the actuator and consequently the needle to a certain adjustable distance.

A spring 1324 (see FIG. 12B) included within injector member 1320 may be configured to bias the actuator back upwards and by that retract also the needle (not shown) so that it does not substantially protrude beyond abutment face 11062 when no force is applied downwards upon the actuator e.g. by a physician.

The outer housing 1322 in this example includes two optional generally axially extending parallel vertical slots 7771v each formed in a respective one of the housing faces 13221 and two optional generally parallel slanted slots 7771s each formed in a respective one of the housing faces 13221. In the cross section of FIG. 12B only one vertical slot 7771v and one slanted slot 7771s is visible. The slanted slots 7771s extend generally parallel to or along axis T and to the needle guide defining axis T (which is here not shown).

The minimal volume connecting tubing 1330, possibly made from silicon, Teflon, thermoplastic PVC (or the like)—may extend within housing 1322 to connect at its lower side to an upstream proximal side of the needle 11077.

The link rod 882 as seen in this embodiment includes two linkage bars 8821. Each linkage bar 8821 as seen couples via a living hinge 21 at its upper side to the pusher 881 and at its lower side to the sliding carriage 883.

A first pin 1111 threaded through a distal lower end of the pusher 881 and through the vertical slots 7771v restricts or constrains movement of the lower end of the pusher 881 along the vertical slots 7771v and a second pin 1112 threaded through the sliding carriage 883 and through the slanted slots 7771s restricts or constrains movement of the sliding carriage 883 along the slanted slots 7771s.

Such positioning of first pin 1111 above the second pin 1112 and to its lateral right hand side hand side, assists in promoting movement of the sliding carriage 883 along a direction generally parallel to axis T.

The needle 11077 includes a bend 87 and is fitted at said bend to the sliding carriage 883 so that movement of the sliding carriage 883 generally along an axis T defined by the generally straight needle guide 11067—is adapted to move the needle's terminal region 24 along same axis T.

The needle may be fitted to the sliding carriage with assistance of the bend to maintain the chamfer/bevel formed at the needle's tip in an orientation where it faces distally away from the abutment face of the injection device towards a body tissue it aims to penetrate as seen e.g. in FIGS. 6A to 6C. It is noted that the needle may alternatively be formed also as a substantially straight needle substantially devoid of any bends therein, while maintaining the desired orientation of the needle may be by fixing the needle to the sliding carriage by other means such as glue (or the like).

Axially urging the pusher 881 in the distal direction is adapted to urge the needle's terminal region via the sliding carriage 883 to advance along axis T to position the needle's distal tip beyond the injection device's abutment face.

Retracting the pusher 881 back up in the proximal direction is adapted to urge the needle's terminal region via the sliding carriage 883 to retract back along axis T to position the needle's distal tip at a position where it does not substantially protrude beyond the injection device's abutment face.

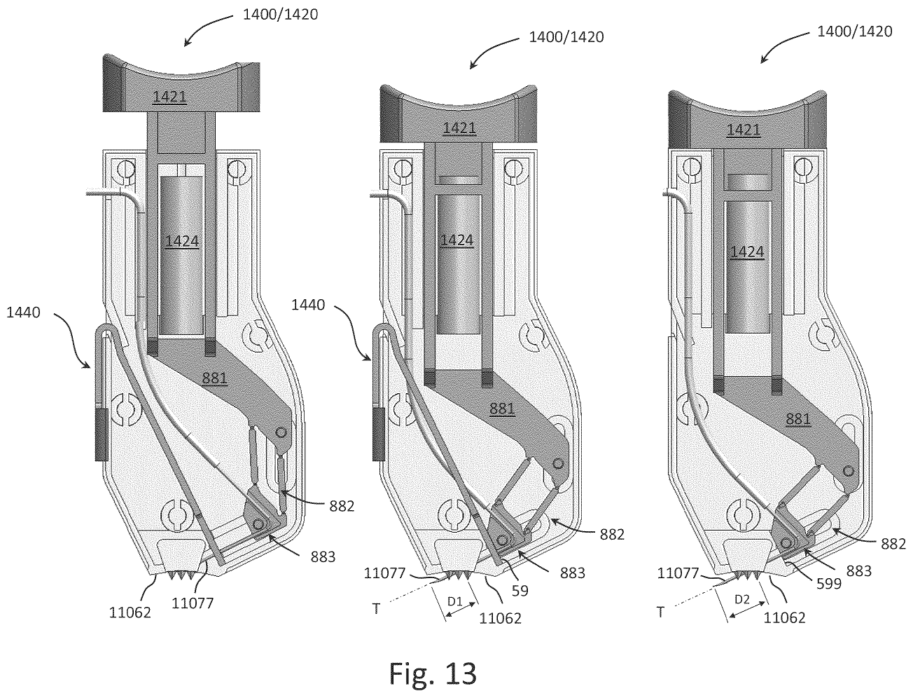

Attention is drawn to FIG. 13 illustrating an embodiment of an injection device 1400 generally similar to the one seen and discussed with respect e.g. to the device seen in FIGS. 12A to 12C, which includes two main separate members, one being a syringe hub member and the other being an injector member, which are fluidly connected by a flexible minimal volume connecting tubing. In these views, only the injector member indicated also by numeral 1420 is seen.

The injector member 1420 may include a needle 11077 that may be formed from relative rigid and inflexible material such as stainless steel. Such a relative rigid needle may be understood as meaning a hypodermic needle, which is commonly used in medical settings and is designed to penetrate the body tissue and/or underlying tissues.

A lower side of the injector member's outer housing is seen including a concave abutment face 11062 from which optionally several barb members may project in the distal direction.

In this embodiment, as in the former embodiment seen in FIG. 12, the injector member's actuating mechanism can be seen having an integral formation in which its actuator 1421, pusher 881, link rod 882 and a sliding carriage 883 are integrally formed as a unitary one piece structure.

The injector member of FIG. 13 as here seen also includes a spring 1424 configured to bias its actuator 1421 back upwards and by that retract also the needle so that it does not substantially protrude beyond abutment face 11062 when no force is applied downwards upon the actuator e.g. by a physician.

In this example, the injector member 1420 can be seen being provided with a removable stopper 1440 for controlling the protrusion of the needle's tip beyond the device's abutment face 11062.

It is noted that in absence of the removable stopper 1440 (see right-hand side of FIG. 13) the needle can protrude a larger distance (see D2 discussed below) beyond the abutment face as defined by the position in which sliding carriage 883 engages against a terminal stop 599 where the device's needle guide (not seen here) opens out towards the sliding cartridge.

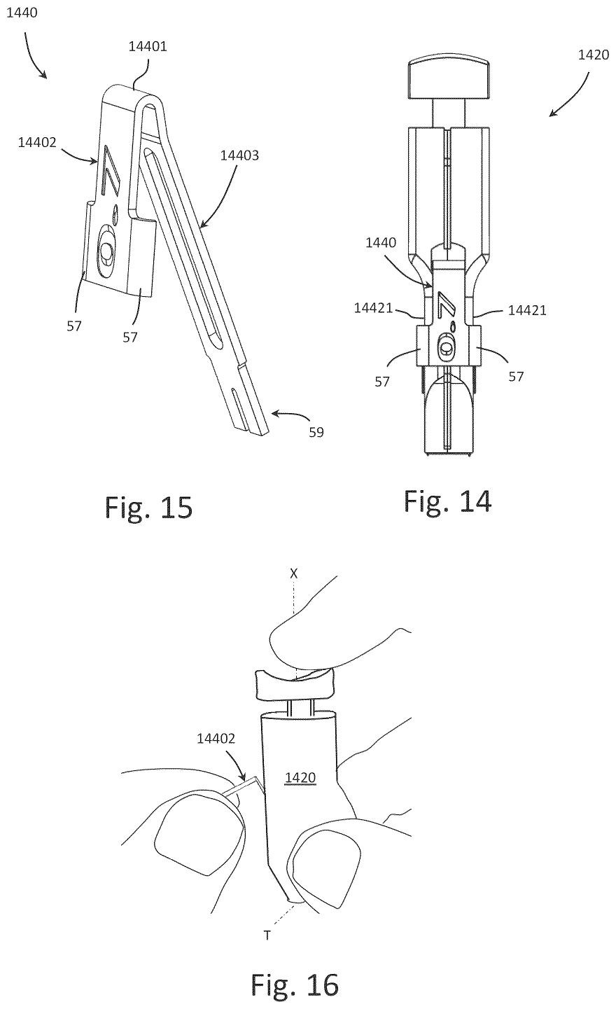

With attention additionally drawn to FIG. 14, the injector member 1420 with the removable stopper 1440 assembled therein can be seen, and with further attention drawn to FIG. 15 the removable stopper 1440 can be seen by itself, possibly after being removed from the injector member.

The removable stopper 1440 as seen in this example includes in a side view a generally V shaped formation having two arms 14402, 14403 of unequal length that meet at an apex 14401. The two arms 14402, 14403 in this example can be flexed one in relation to the other generally about the apex.

The longer arm 14403 can be seen extending to an interface 59 at its free end, which is designed to engage with the sliding carriage 883 during use in order to limit the distance that the device's needle can protrude beyond the abutment face. The shorter arm 14402 is deigned to remain outside of the injector member when the removable stopper 1440 is assembled to the injector member, and includes two tabs 57 adjacent its free end.

As seen in FIG. 14, these tabs 57 may be designed to protrude slightly sideways beyond the housing faces 14421 of the injector member (see also housing face 13221 indicated in FIG. 12A) to ease grasping onto these tabs in order to remove the removable stopper from the injector member when needed.

Attention is drawn to FIG. 16 showing an example where the injector member 1420 is held by a thumb and a finger (e.g. index finger) of one hand, while the tabs of the removable stopper's shorter arm 14402 are held by a thumb and a finger (e.g. index finger) of the other hand.

In this example, the shorter arm 14402 of the removable stopper can be seen being flexed slightly outwards away from the device by flexing it away from the removable stopper's longer arm (which is still within the device), in order to assist in removing the removable stopper from the injector member.

Attention is drawn back to FIG. 13. In the left-hand side view of FIG. 13, the needle 11077 can be seen in a retracted position not protruding beyond the abutment face 11062.

In the middle view of FIG. 13, the needle 11077 can be seen being urged to advance along axis T and protrude a first distance D1 beyond the abutment face 11062, which is defined by the position in which the sliding carriage 883 engages interface 59 of the removable stopper 1440.

Designing the removable stopper to substantially avoid ‘tolerance stack-up’—by configuring the removable stopper to engage the needle adjacent to where it is designed to protrude out of the injection device (here via engagement between interface 59 and sliding carriage 883)—provides a higher degree of certainly that the needle will protrude from the device's abutment face the distance D1 that was intended and designed.

In the right-hand side view of FIG. 13 it can be seen that after altering the position of the removable stopper 1440, in this optional example by removing the removable stopper 1440 from the device, the needle 11077 can be urged to further protrude beyond the abutment face 11062 to reach a second distance D2, which is larger than the first distance D1.

Provision of such two controlled pre-defined distances D1 and D2 for the needle to protrude beyond the device's abutment face, may assist a physician in targeting a desired targeted layer within the eye that the device is aimed at. This may be accomplished by first attempting to reach such targeted layer at distance D1 and if the needle's tip has not yet reached such targeted layer, the second distance D2 can be tested.

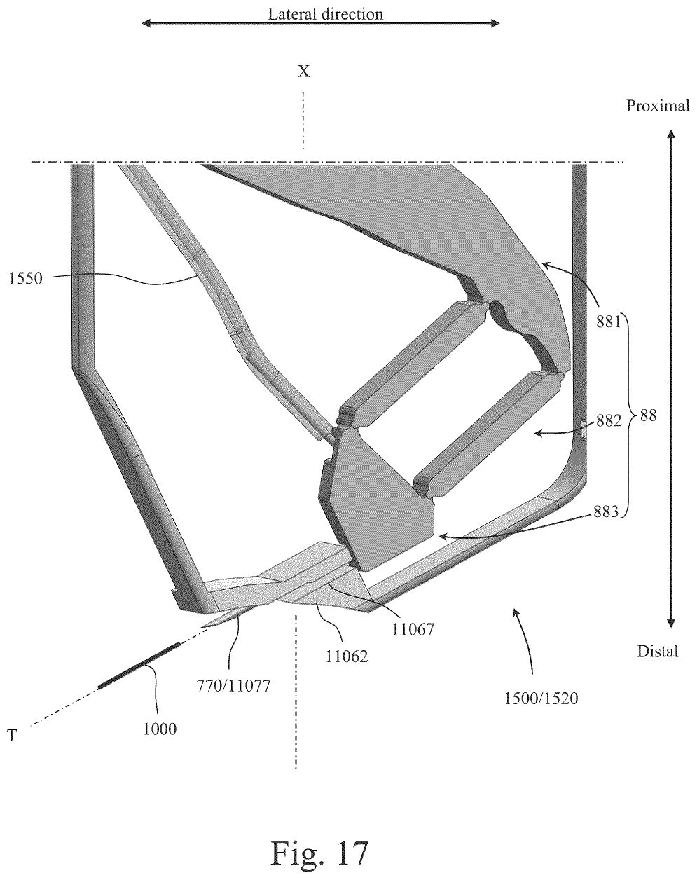

Attention is drawn to FIG. 17 schematically showing an embodiment of an injection device 1500 suitable for administrating a substance and/or introducing an implant towards a target site within a patient's body tissue. The portion of injection device 1500 seen in this figure may also be referred to as an injector member 1520 of the device in cases where the injection device is formed from two separate members, such as in the examples illustrated in FIGS. 10A, 10B and 12.

In this figure a cross sectional view of a terminal distal region of the injection device is shown revealing the interior of the device's housing. In particular here an actuating mechanism 88 can be seen including its pusher 881, link rod 882 and sliding carriage 883, which in this example are formed having an integrally formed unitary one piece structure.

A minimal volume connecting tubing 1550 can be seen being fitted at its lower side to an upstream proximal side of a delivery needle 770 designed for introducing an implant 1000 towards a target site within patient's body tissue. In this example the delivery needle 770 may be the same needle 11077 used in former described embodiments for administrating substances (such as medications) towards the target site. The minimal volume connecting tubing 1550 may be connected at its upper side to a syringe member of or associated with the injection device (both not shown).

In certain cases, the needle(s) 770/11077 may be formed from relative rigid and inflexible material such as stainless steel. Such a relative rigid needle may in some cases be a hypodermic needle, which is commonly used in medical settings and is designed to penetrate the body tissue and/or underlying tissues. In other cases, the needle may be formed from more flexible materials such as nitinol (or the like).

The needle 770/11077 is fitted here at or adjacent its bend to the sliding carriage 883 so that movement of the sliding carriage 883 generally along an axis T defined by the needle guide 11067—is adapted to move a terminal region of the needle along same axis T to protrude beyond an axial distal facing abutment face 11062 of the device. Axes T as seen may be inclined to the injection device's longitudinal axis X.

In this example, the injection device can be seen being configured to introduce the implant 1000 accordingly through needle 770/11077 along axis T towards the target site within a patient's body tissue, such as towards and into a suprachoroidal space (SCS) within an eye.

The injection device can be seen being configured to eject the implant out of the distal tip of the needle using hydraulic actuation. This may be performed by using an implant introducer in form of a syringe for injecting fluid via tubing 1550 that enters the needle to thereby push and eject the implant 1000 out of the needle's tip. Loading of the implant 1000 into a parked position within the needle may be accomplished in this example by pushing and urging the implant upstream into the needle through its distal tip.

Fluids suitable for ejecting implant 1000 out of the needle's tip may be chosen to have relatively high viscosity, and in some cases may be chosen to be of a non-Newtonian type whose viscosity (resistance to flow) changes depending on the applied stress or strain rate applied thereupon. As a result, such non-Newtonian fluids may more easily flow through small-diameter tubes, such as the minimal volume connecting tubing 1550 where flow rates and shear rates can be high.

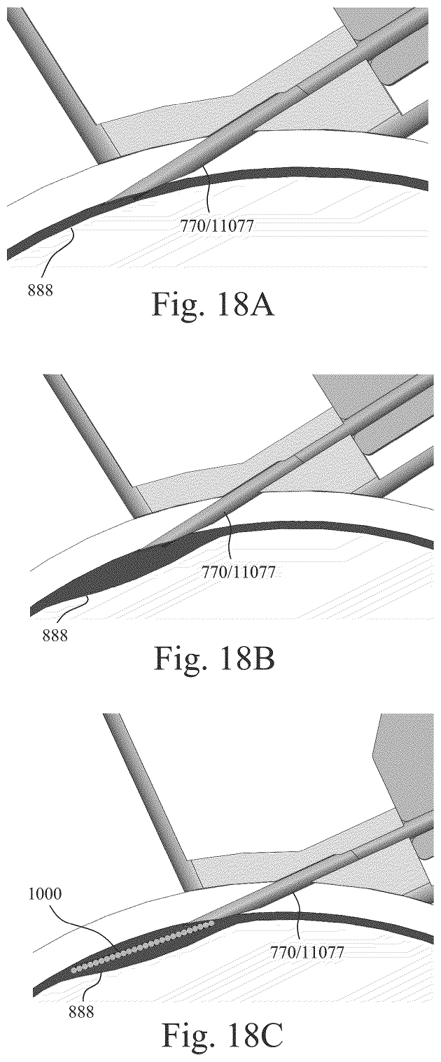

Attention is drawn to FIGS. 18A to 18C showing an injection device such as those seen in any one of the embodiments of FIGS. 17 to 24, during different stages of a minimally invasive procedure here illustrated as optionally aimed at a target site of the suprachoroidal space (SCS) 888.

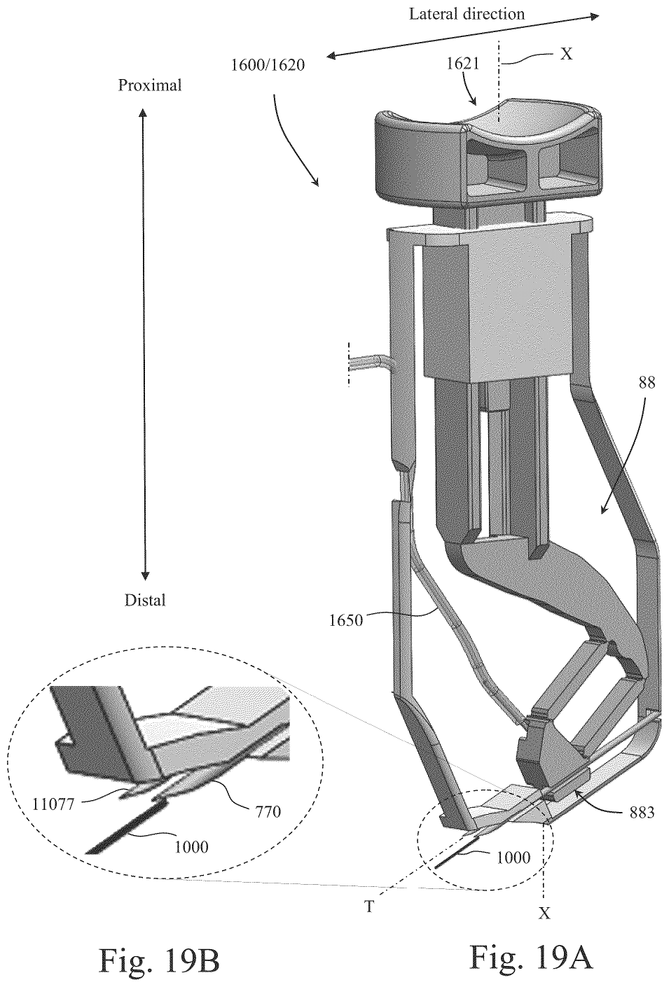

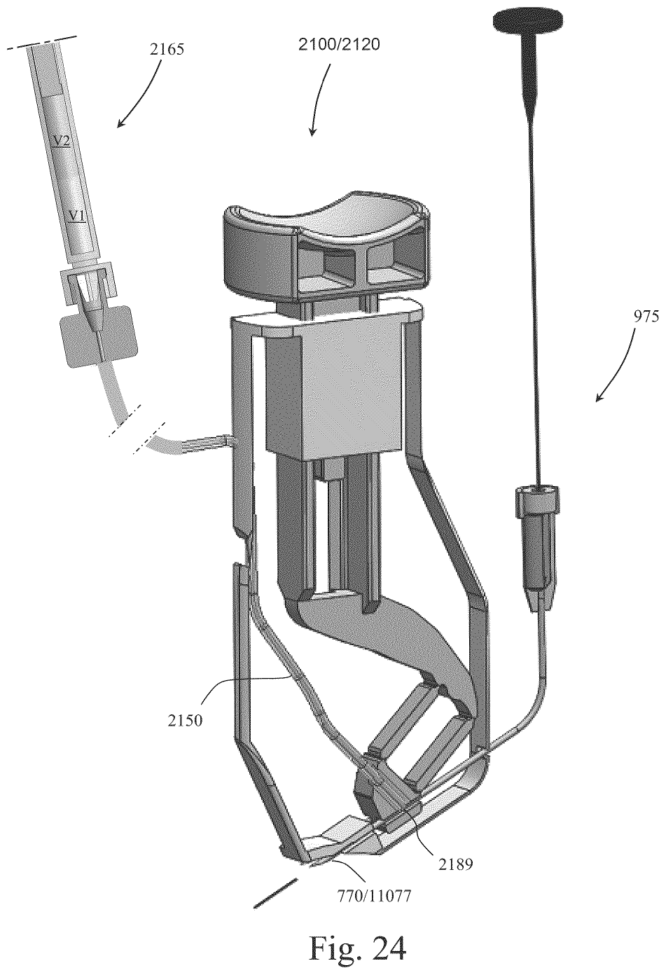

In these FIG. 18, the visible needle is represented by numerals 770 and 11077. Consequently, it can be inferred that a single needle, as shown in the embodiments of FIGS. 17 and 24, may serve both as a delivery needle 770 for introducing an implant to the target site and as a needle 11077 for administering substances (e.g., medications) to the same site. Additionally, the use of numerals 770 and 11077 to indicate the visible needle in FIG. 18 may suggest that two separate needles may be employed (as seen in FIGS. 19 to 23), with only one being visible in these figures. In this case, one needle 770 functions as the delivery needle for an implant, while the other needle 11077 is used as a secondary needle for administering substances (such as medications) to the same target site.

It is noted that in injection device embodiments disclosed herein in which two needles 770, 11077 are used, the needles may not necessarily be similar. For instance, the needles 770, 11077 may have different diameters and/or may be formed from different materials and/or may have different tip geometries (and the like).

In FIG. 18A, the needle(s) can be seen reaching the target site, similar to the depiction in FIG. 6 of this disclosure. In this example, the target site is the suprachoroidal space (SCS) 888. In the next step, shown in FIG. 18B, a liquid (such as saline, gel or a similar substance) may be introduced via the needle (or one of the needles 11077) into the target site, causing the suprachoroidal space (SCS) 888 to hydro separate, inflate, open, or expand. Finally, in FIG. 18C, an implant 1000 may be introduced through the needle (or the other needle 770) into the expanded suprachoroidal space (SCS) 888.

It is noted that the steps shown in these FIGS. 18A to 18C may not necessarily be performed in sequence. For instance, the steps seen in FIGS. 18B and 18C may be formed substantially together or may at least partially overlap (e.g. expanding a target site may occur while inserting an implant therein).

Attention is drawn to FIGS. 19A and 19B schematically showing another embodiment of an injection device 1600 suitable for administrating a substance and/or introducing an implant towards a target site within a patient's body tissue. Here the portion of injection device 1600 seen is its injector member 1620.

Similar to FIG. 17, this illustration presents a cross-sectional view of the injection device, exposing the interior of its housing and a manually activated actuator 1621 with its actuating mechanism 88. Once again, the actuating mechanism is optionally depicted in this figures as having an integrally formed, unitary, one-piece structure.

As best seen in the enlarged view of FIG. 19B, injection device 1600/1620 includes two needles attached to a carriage 883 of its actuating mechanism 88. One of the needles 11077, to which the minimal volume connecting tubing 1650 is attached, acts as a secondary needle for administrating substances towards the target site, and the other needle being the delivery needle 770, which is designed to introduce an implant 1000 towards the target site.

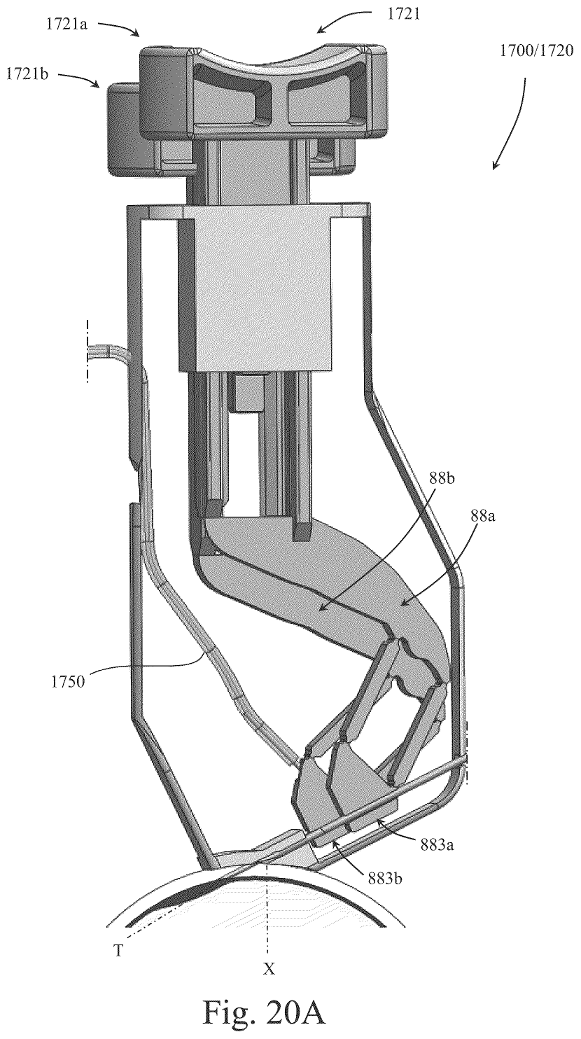

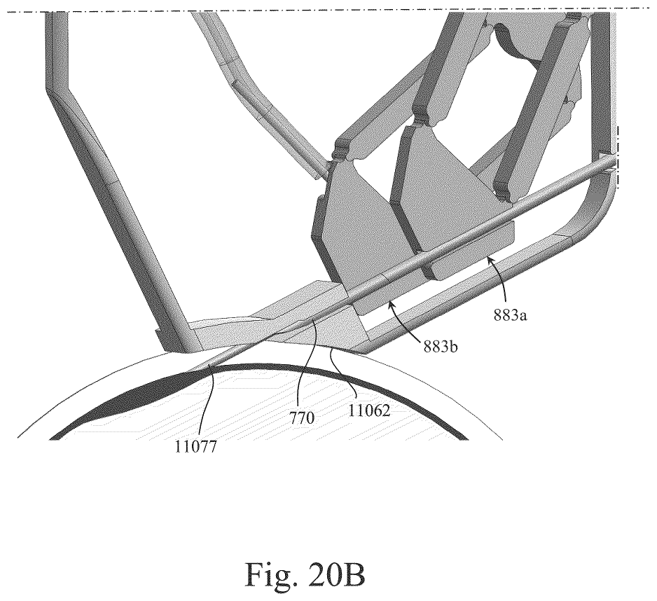

Attention is drawn to FIGS. 20A and 20B schematically showing another embodiment of an injection device 1700 suitable for administrating a substance and/or introducing an implant towards a target site within a patient's body tissue. Here the portion of injection device 1700 seen is its injector member 1720.

Similar to FIG. 17, this illustration presents a cross-sectional view of the injection device, exposing the interior of its housing. In this example, the injection device is seen including an actuator system 1721 comprised of two independently controlled sub actuators 1721a, 1721b. Each sub actuator 1721a, 1721b is configured to activate a respective distinct actuating mechanism 88a, 88b, here also being each depicted as having an integrally formed, unitary, one-piece structure.

As best seen in the enlarged view of FIG. 20B, injection device 1700/1720 includes two needles each being attached to a different actuating mechanism 88a, 88b and therefore to a different carriage 883a, 883b of its actuating mechanism. One of the needles 11077, to which the minimal volume connecting tubing 1750 is attached, acts as a secondary needle for administrating substances towards the target site, and the other needle being the delivery needle 770, which is designed to introduce an implant towards the target site.

As a result, in injection device 1700/1720 the needles 770, 11077 can be activated to move independently one in relation to the other as they are attached to different carriages 883a, 883b. Such independent activation of the needles 770, 11077 may allow flexibility in advancing the needles e.g. to protrude different distances beyond device's abutment face 11062.

For example, in certain cases, the ability to independently control the movement of delivery needle 770 may allow a user operating the injection device to advance the delivery needle 770 slightly deeper into a suprachoroidal space (SCS) after it has been previously expanded (see, for instance, the expansion depicted in FIGS. 18B and 18C).

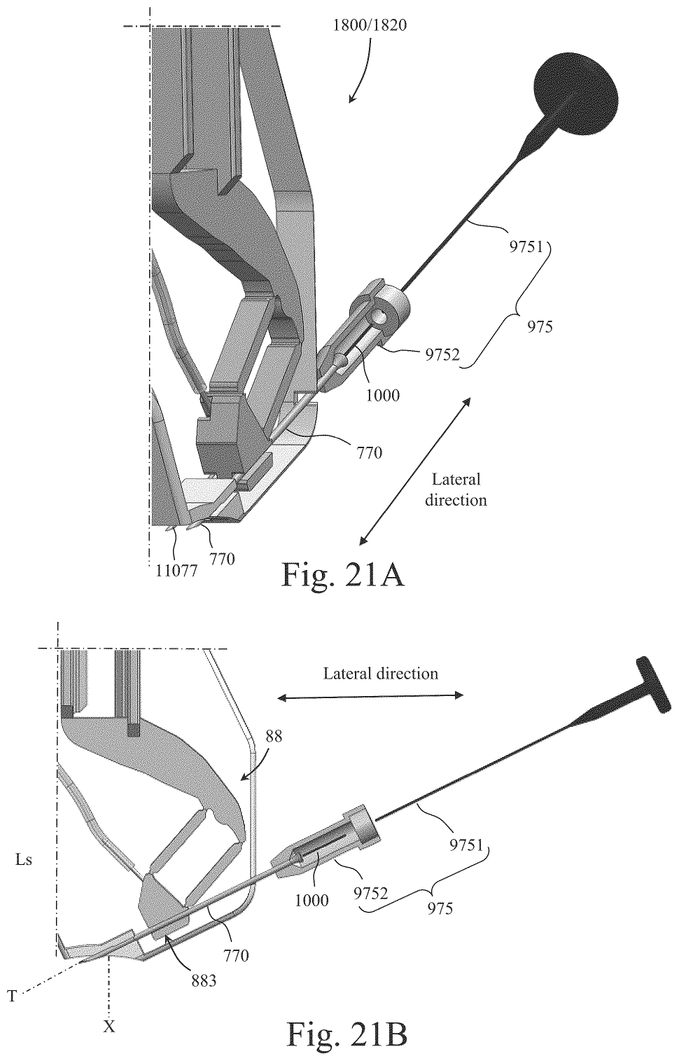

Attention is drawn to FIGS. 21A and 21B schematically showing an embodiment of an injection device 1800 suitable for administrating a substance and/or introducing an implant towards a target site within a patient's body tissue. Here the portion of injection device 1800 seen is its injector member 1820.

Injection device 1800, 1820 is defined having a given lateral side ‘Ls’ towards which axis T slants, and in these figures the device 1800, 1820 can be seen including an implant introducer 975 that extends in a general opposing lateral direction to the given lateral side ‘Ls’ at a distal portion of the device.

The implant introducer 975 includes a pusher 9751 (such as a metal wire) and a loading chamber 9752, positioned between the pusher 9751 and the delivery needle 770. The implant 1000 is housed/parked within the loading chamber 9752 and can be advanced for ejection out of the distal tip of the needle 770 by pushing it forward with the pusher 9751 into the needle, while guiding it out of the needle towards the needle's tip.

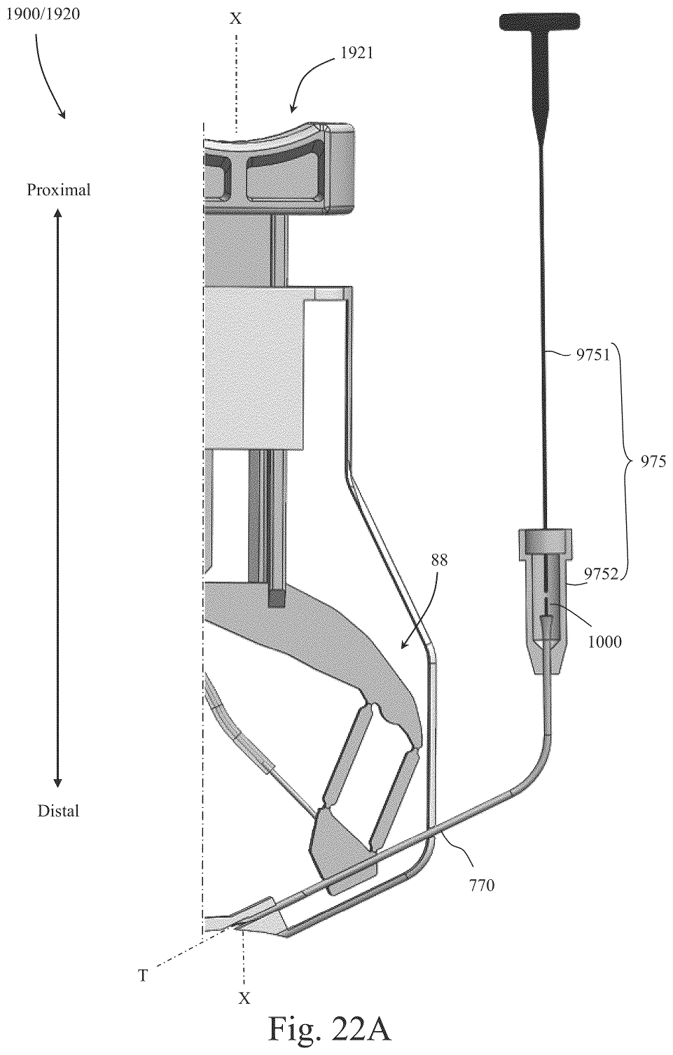

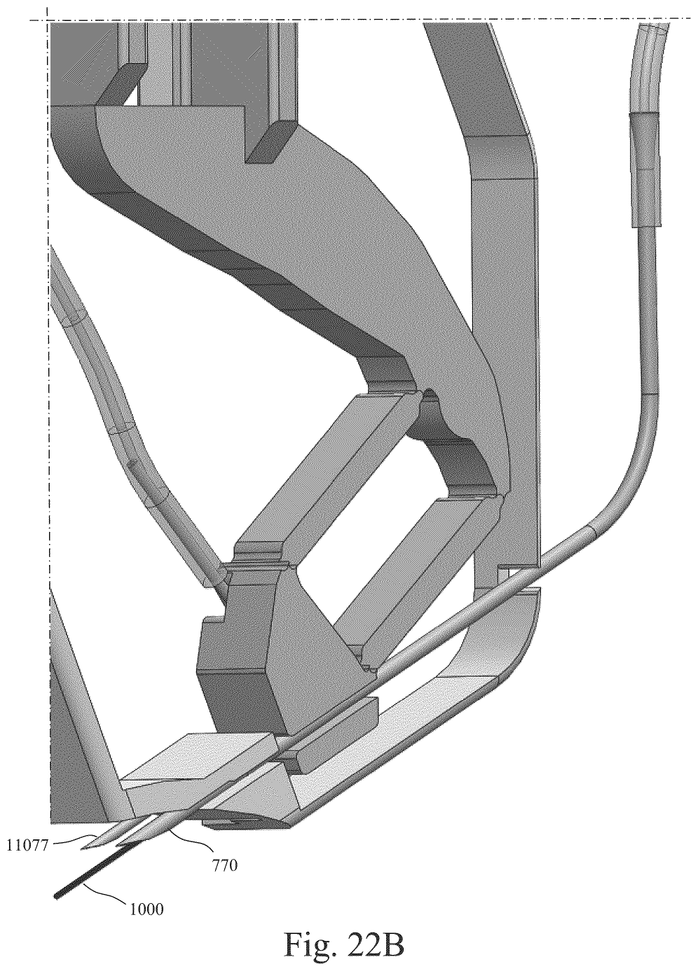

Attention is drawn to FIGS. 22A and 22B schematically showing an embodiment of an injection device 1900 suitable for administrating a substance and/or introducing an implant towards a target site within a patient's body tissue. Here the portion of injection device 1900 seen is its injector member 1920.

The injection device 1900, 1920 features a generally axially extending implant introducer 975, which comprises a pusher 9751 (such as a metal wire) and a loading chamber 9752, positioned between the pusher 9751 and the delivery needle 770.

In this configuration, needle 700 is shown with a curved trailing end, bending upward to facilitate access to the pusher 9751 in a more intuitive way from the same proximal side from which the device's actuator is operated (and hence possibly by the same fingers or hand of the user).

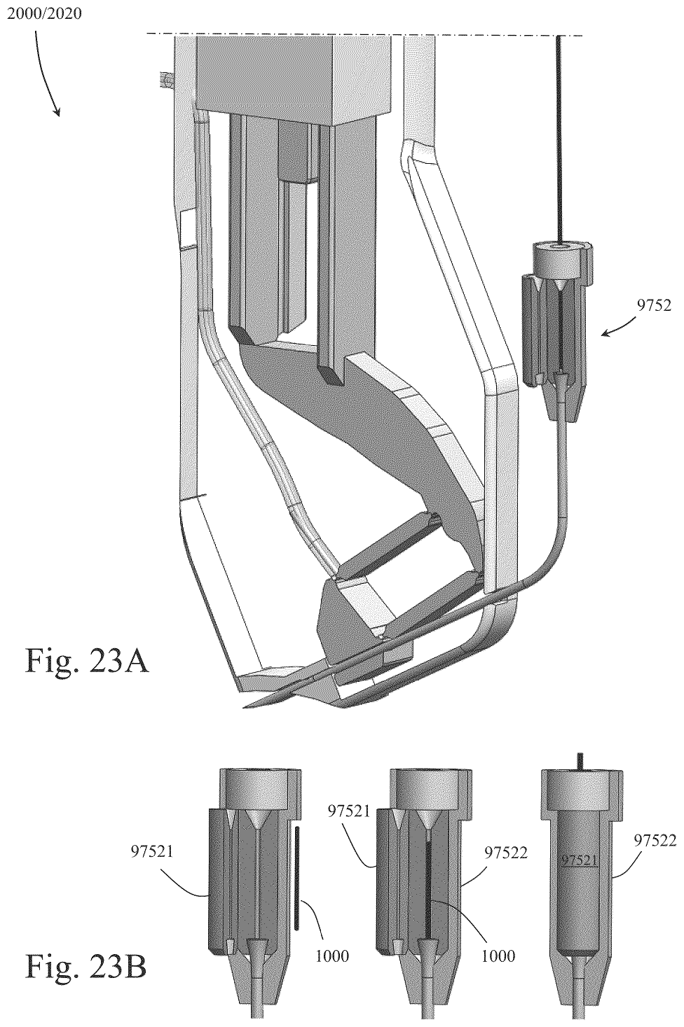

Attention is drawn to FIG. 23A schematically showing an embodiment of an injection device 2000 generally similar to that in FIG. 22, which includes an implant loading chamber 9752. Here the portion of injection device 2000 seen is its injector member 2020. In FIG. 23B the implant loading chamber can be seen during different stages of loading an implant 1000 therein.

The loading chamber 9752 includes a base 97522 and a movable flap 97521, which can be adjusted to expose the interior of the loading chamber 9752 for implant 1000 insertion. Once the implant 1000 is placed on the base 97522, the flap 97521 can be moved to a closed position, securely enclosing the implant 1000 and preparing it for use.

In the various embodiments illustrated in FIGS. 17 to 24, an implant 1000 may be loaded into a parked position within the injection device in one of two ways: either by pushing it upstream into the needle 11077/770 through its distal open tip or, in cases where the device includes a loading chamber 9752, by placing the implant directly into the chamber for storage until use. Additionally, in both loading methods, multiple implants can be parked within the device. For instance, when using a loading chamber 9752, a first implant can be placed inside the chamber and advanced into the needle using the pusher 9751. Subsequently, additional implants can be sequentially loaded into the chamber and parked within the trailing end of the needle in the same manner.

Attention is drawn to FIG. 24 schematically showing another embodiment of an injection device 2100 suitable for administrating a substance and/or introducing an implant towards a target site within a patient's body tissue. Here the portion of injection device 2100 seen is its injector member 2120.

In this embodiment, similar to the embodiment seen in FIG. 17, the delivery needle 770 may be the same needle 11077 used for administrating substances towards the target site.

Here however, the minimal volume connecting tubing 2150 is shown attached at its lower side to the needle at a junction 2189, where the implant introducer 975 is also connected.

Prior to use, the implant 1000 may be loaded into the needle from its distal tip to a position in between the needle's tip and the junction 2189, and a syringe 2165 may be coupled to an upstream of the connecting tubing 2150 for providing liquid substances towards the needle. Possibly, the implant 1000 may be parked within a loading chamber as previously discussed.

The syringe's barrel may be filled with two types of fluids having different viscosities V1 and V2, where the viscosity V1 of the fluid located at the leading side of the syringe may be lower than the viscosity of the fluid located at the trailing side of the syringe.

The procedure depicted in FIGS. 18A to 18C can be performed using the injection device 2100, 2120 as follows. Once the needle's tip is positioned at the target site (as shown in FIG. 18A), the lower viscosity fluid V1 may be introduced, flowing past the implant 1000, which remains inside the needle, and filling the suprachoroidal space (SCS) 888 (as shown in FIG. 18B). The low viscosity V1 allows the fluid to bypass the implant, keeping it substantially in place within the needle.

Subsequently, the higher viscosity fluid V2 may be dispensed from the syringe, exerting force on the implant, which it cannot bypass, thereby pushing the implant 1000 out of the needle's tip into the expanded suprachoroidal space (SCS) 888 (as seen in FIG. 18C).

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

Further more, while the present application or technology has been illustrated and described in detail in the drawings and foregoing description, such illustration and description are to be considered illustrative or exemplary and non-restrictive; the technology is thus not limited to the disclosed embodiments. Variations to the disclosed embodiments can be understood and effected by those skilled in the art and practicing the claimed technology, from a study of the drawings, the technology, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage.

The present technology is also understood to encompass the exact terms, features, numerical values or ranges etc., if in here such terms, features, numerical values or ranges etc. are referred to in connection with terms such as “about, ca., substantially, generally, at least” etc. In other words, “about 3” shall also comprise “3” or “substantially perpendicular” shall also comprise “perpendicular”. Any reference signs in the claims should not be considered as limiting the scope.

Although the present embodiments have been described to a certain degree of particularity, it should be understood that various alterations and modifications could be made without departing from the scope of the invention as hereinafter claimed.

1. A method of introducing an implant into a target site within a body tissue, the method comprising the steps of:
providing an injection device comprising a needle and an abutment face,
placing the abutment face upon an outer side of a body tissue, and
urging the needle to advance beyond the abutment face towards the target site within the body tissue, wherein
introducing the implant towards the target site comprises urging the implant through the needle to be ejected out of the needle at its distal tip.

2. The method of claim 1, wherein prior to urging the implant to be ejected out of the needle the implant is located within the needle.

3. The method of claim 2, wherein implant is located adjacent the distal tip of the needle.

4. The method of claim 3, wherein locating the implant within the needle is by urging the implant into the needle via its distal tip.

5. The method of claim 2, wherein urging the needle to be ejected out of the needle is performed by hydraulic power.

6. The method of claim 2, wherein urging the needle to be ejected out of the needle is by mechanically pushing it out of the needle, for example by a push wire.

7. The method of claim 1 and comprising an additional secondary needle for injecting liquid substances towards the target site.

8. The method of claim 7, wherein the needle through which the implant moves is a delivery needle and terminal sections of both the delivery and the secondary needle extend generally one alongside the other.

9. The method of claim 8, wherein the injection device being arranged to move the delivery and the secondary needle together.

10. The method of claim 8, wherein the injection device being arranged to move the delivery and the secondary needle in an independent manner one from the other.

11. An injection device comprising an abutment face, a needle being movable to protrude beyond the abutment face and the injection device comprises an implant introducer for urging an implant out of a distal tip of the needle.

12. The injection device of claim 11, wherein the implant is parked within the injection device prior to being urged out of the distal tip of the needle.

13. The injection device of claim 11, wherein the implant introducer comprises a syringe for applying hydraulic power against the implant.

14. The injection device of claim 11, wherein the implant introducer comprises a pusher for applying mechanical force against the implant.

15. The injection device of claim 14 and having a longitudinal extension extending along an axis X and the abutment face faces the distal axial direction, wherein the implant introducer extends generally laterally away from a distal portion of the injection device.

16. The injection device of claim 14 and having a longitudinal extension extending along an axis X and the abutment face faces the distal axial direction, wherein the implant introducer extends generally along axis X.

17. The injection device of claim 16 and comprising a secondary needle for injecting liquid substances towards the target site.

18. An injection device comprising an abutment face, and a pair of generally parallel needles being movable to protrude beyond the abutment face.

19. The injection device of claim 18, wherein one of the needles is configured to deliver an implant out of its distal tip.

20. The injection device of claim 19, wherein the other needle is configured to inject liquid substances out of its distal tip.
